(19) United States
(12) Patent Application Publication
(10) Pub. No.: US 2018/0296460 A1
(43) Pub. Date: Oct. 18, 2018

(54) LIP PRODUCT FORMULATION, APPLICATOR, AND METHOD FOR MAKING SAME

(71) Applicant: EOS PRODUCTS, LLC, New York, NY (US)

(72) Inventors: Mike Wong, East Windsor, NJ (US); JoAnn Hansen, Shrewsbury Township, NJ (US); Shanta Benyarko, Burlington, NJ (US); Bhavini Seth, East Windsor, NJ (US)

(21) Appl. No.: 15/487,057

(22) Filed: Apr. 13, 2017

(51) Int. Cl. A61K 8/88; A61Q 19/00; A61K 8/92; A61K 8/33; A45D 40/00

(52) U.S. Cl. CPC A61K 8/88; A61Q 19/001; A61K 8/922; A61K 8/33; A45D 2040/0012; A61K 2800/30; A61K 2800/874; A61K 2800/594; A45D 40/00

(57) ABSTRACT

Provided are lip-based compositions such as lipsticks, lip balms, and lip glosses that have favorable transparency, haze, temperature stability, hardness, taste, payoff, moisturizing effect, feel, and/or resistance to droop and/or shear. In certain embodiments, the lip-based compositions are for use with a container that exposes a substantial percentage of the surface area of the lip-based composition. Also provided are processes for production of such lip-based compositions.

### FIELD OF THE DISCLOSURE

The present disclosure relates generally to lip-based compositions such as lipsticks, lip balms, and lip glosses that have favorable appearance, transparency, haze, temperature stability, hardness, payoff, glide resistance, moisturizing effect, texture, taste, odor and/or resistance to droop and/or shear. In certain embodiments, the lip-based compositions are for use with applicators that expose a substantial percentage of the total surface area of the lip-based composition, for example, applicators that comprise no moving parts or non-telescoping. Also provided are processes for production of such lip-based compositions.

### BACKGROUND

Previous attempts at lip products such as lipsticks, lip balms, and lip glosses that have a high level of transparency with high transmittance and/or low haze have either lacked stiffness, tended to dry-out and clump or crack on the lip, or lacked the ability to effectively treat or prevent lip-related conditions e.g., dried, cracked, chapped lips, etc.). Attempts to solve these problems have lead to formulations that were difficult to spread on the lips, had unpleasant tactile properties (e.g., heavy, oily, tacky, etc.), tasted bad, and had limited beneficial effects for lip-related conditions. Moreover, such formulations were generally intended to be used in containers wherein only a relatively small percentage of the surface area of the lip balm, for example the applicating surface area of a mound or stick of lip balm, was exposed for application (e.g. tubes and/or jars). Such formulations do not have the requisite temperature stability, hardness, and/or resistance to droop and/or shear for use with a container that exposes a substantial percentage of the total surface area of the lip-based composition. An example of a lip balm container that exposes a substantial percentage of the surface area of the lip-based composition is described in U.S. Pat. No. 8,888,391 (hereinafter, “Teller”), entitled Lip balm with Spherical Surface and Method for Producing, which is incorporated herein by reference in its entirety.

Moreover, previous attempts at formulating a lip balm that has high transparency are often very soft, and they exhibit such undesirable properties as syneresis (sweating), haziness, and low “payoff” (i.e., the product deposits an insufficient amount of the product on the skin when used).

Production of a lip balm that has favorable appearance, transparency, transmittance, haze, temperature stability, hardness, taste, payoff, moisturizing effect, feel, taste, odor and/or resistance to droop and/or shear that can also be used with a container that exposes a substantial percentage of the surface area of the lip-based composition is therefore desirable. The present disclosure describes exemplary embodiments of formulations that may meet various combinations of one or more of these features, as well as processes for their production.

### SUMMARY

Certain embodiments may provide, for example, compositions, articles, and methods relating to self-supporting (for example self-supporting at room temperature), transparent lip balm. In certain embodiments, for example, the lip balm composition may comprise a combination of one or more structuring agents, emollients, moisturizers, oils, essential oils, lipids, thickening agents, stability agents, shine enhancers, natural flavors, artificial flavors, odorants, sunscreen agents (for example uv blocking agents), cosmetic ingredients, and/or antioxidants.

Certain embodiments may provide, for example, a lip balm composition comprising 60-85 wt. % of at least one emollient, no more than 40 wt. % (for example less than 40 wt. % or 10-40 wt. %) of at least one structuring agent (for example polyamide-3 and/or polyimide-8), optionally at least one silicon-containing compound (for example fumed silica and/or silica dimethyl silylate; for example 0.25-2 wt. % of at least one silicon-containing compound), wherein the lip balm composition may have a transmittance of at least 80% after 24 hours according to the Transmittance Test described hereunder, a hardness in the range of 16-24 mm according to the Hardness Test described hereunder, a payoff in the range of 0.2-0.6 grains according to the Payoff Test described hereunder, and a moisture retention of at least 70 (for example at least 90) after 15 minutes according to the Moisture Retention Test described hereunder.

In certain embodiments, for example, the lip balm composition may comprise less than 40 wt. % of the at least one structuring agent. In certain further embodiments, for example, the lip balm composition may comprise less than 30 wt. % polyamides. In certain embodiments, for example, the lip balm composition may comprise less than 30 wt. % dicaprylyl carbonate. In certain embodiments, for example, the lip balm composition may be anhydrous.

In certain embodiments, for example, the lip balm composition may comprise one or more of ricinus communis (Castor) seed oil, dicaprylyl carbonate, decyl isostearate, butyrospermum parkii (shea) oil, pentaerythrityl tetraisostearate, isostearyl isostearate, octyldodecanol, polyamide-3, polyamide-8, and fumed silica or silica dimethyl silylate. In certain further embodiments, for example, the lip balm composition may comprise one or more of cocas nucifera (coconut) oil, helianthus annuus (sunflower) seed oil, aloe barbadensis leaf juice, persea gratissima (avocado) oil, and one or more flavorants (for example flavor FZ9255 and/or sweetness enhancer FZ7492).

In certain embodiments, for example, the lip balm composition may comprise in the range of 10-30 wt. % ricinus communis (Castor) seed oil, in the range of 10-50 wt. % dicaprylyl carbonate, in the range of 0-20 wt. % decyl isostearate, in the range of 0-20 wt. % butyrospermum parkii (shea) oil, in the range of 0-20 wt. % pentaerythrityl tetraisostearate, in the range of 2-20 wt. % isostearyl isostearate, in the range of 0-10 wt. % octyldodecanol, in the range of 10-20 wt. % polyamide-3, in the range of 2-10 wt. % polyamide-8, and in the range of 0.25-2 wt. % silica dimethyl silylate or fumed silica.

In certain embodiments, for example, the lip balm composition may comprise in the range of 15-25 wt. % ricinus communis (Castor) seed oil, in the range of 11-17 wt. % dicaprylyl carbonate, in the range of 10-15 wt. % decyl isostearate, in the range of 7-13 wt. % butyrospermum parkii (shea) oil, in the range of 7-13 wt. % pentaerythrityl tetraisostearate, in the range of 2-6 wt. % isostearyl isostearate, in the range of 2-6 wt. % octyldodecanol, in the range of 10-20 wt. % polyamide-3, in the range of 2-10 wt. % polyamide-8, and in the range of 0.25-2 wt. % silica dimethyl silylate. In certain embodiments, for example, the lip balm composition may comprise about 20 wt. % ricinus communis (Castor) seed oil, about 14 wt. % dicaprylyl carbonate, about 12.75 wt. % decyl isostearate, about 10 wt. % butyrospermum parkii (shea) oil, about 10.7 wt. % pentaerythrityl tetraisostearate, about 4.25 wt. % isostearyl isostearate, about 4 wt. % octyldodecanol, about 15 wt. % polyamide-3, about 5 wt. % polyamide-8, and about 1 wt. % silica dimethyl silylate. In certain further embodiments, for example, the lip balm composition may pass one or more (for example all) of the Appearance Test, the Color Test, Odor Test, and the Texture Test, each of which is described herebelow. In certain embodiments, for example, the lip balm may have a haze value of less than 10% (for example less than 5%, less than 3%, less than 1%, less than 0.5%, or less than 0.1%) according to ASTM D1003. In certain further embodiments, for example, the lip balm composition may further comprise one or more additional flavorants, odorants, and/or emollients. For example, the lip balm composition may further comprise about 0.1 wt. % cocos nucifera (coconut) oil, about 0.1 wt. % helianthus annuus (sunflower) seed oil, about 0.1 wt. % persea gratissima (avocado) oil, about 1 wt. % flavor FZ9255, and about 2 wt. % sweetness enhancer 177492.

In certain embodiments, for example, the lip balm composition may comprise in the range of 15-25 wt. % ricinus communis (Castor) seed oil, in the range of 45-55 wt. % dicaprylyl carbonate, in the range of 5-10 wt. % isostearyl isostearate, in the range of 0-5 wt. % octyldodecanol, in the range of 10-20 wt. % polyamide-3, in the range of 2-10 wt. % polyamide-8, and in the range of 0.25-2 wt. % fumed silica. In certain embodiments, for example, the lip balm composition may comprise about 15 wt. % ricinus communis (Castor) seed oil, about 49 wt. % dicaprylyl carbonate, about 10 wt. % isostearyl isostearate, about 15 wt. % polyamide-3, about 5 wt. % polyamide-8, and about 1 wt. % fumed silica. in certain further embodiments, for example, the lip balm composition may pass one or more (for example all) of the Appearance Test, the Color Test, Odor Test, and the Texture Test, each of which is described hereunder. In certain embodiments, for example, the lip balm may have a haze value of less than 10% (for example less than 5%, less than 3%, less than 1%, less than 0.5%, or less than 0.1%) according to ASTM D1003.

In certain embodiments, for example, the lip balm composition may be anhydrous, wax-free, PEG-free, PPG-free, silicone-free, preservative-free, volatiles-free, volatile alcohol-free, or a combination of two or more thereof.

In certain embodiments, for example, the lip balm composition may pass the Appearance Test described herein. In certain embodiments, for example, the lip balm composition may pass the Color Test described hereunder. In certain embodiments, for example, the lip balm composition may pass the Odor Test described herein. In certain embodiments, for example, the lip balm composition may pass the Texture Test described herein. In certain embodiments, for example, the lip balm may have a haze value of less than 10% (for example less than 5%, less than 3%, less than 1%, less than 0.5%, or less than 0.1%) according to ASTM D1003.

Certain embodiments may provide, for example, a method of making a self-supporting, transparent lip balm composition, comprising heating at least one emollient to a temperature above 85° C., adding at least one solid silicon compound (for example a non-silicone silicon compound) and/or at least one structuring polymer to the at least one emollient to form a mixture, cooling the mixture to a temperature below 80° C., and introducing at least one flavorant.

Certain embodiments may provide, for example, a method of making a quantity of lip balm, comprising heating a first quantity of at least one emollient to a temperature in the range of 90-95° C. in an agitated process vessel, followed by introducing a quantity of at least one solid silicon compound (for example silica dimethyl silylate and/or fumed silica) to the heated first portion, followed by introducing a quantity of at least one structuring polymer to the agitated process vessel, followed by introducing a second quantity of at least one emollient to the agitated process vessel, followed by cooling the agitated process vessel to a temperature in the range of 78-82° C., followed by introducing a quantity of at least one flavorant to the agitated process vessel. In certain embodiments, for example, introducing the quantity of at least one solid silicon compound and introducing the quantity of at least one structuring polymer may each comprise mixing for at least 10 minutes or until the contents of the process vessel are transparent. In certain embodiments, for example, introducing the quantity of at least one solid silicon compound and introducing the quantity of at least one structuring polymer may each comprise mixing under conditions sufficient to produce a vortex in the agitated process vessel. In certain embodiments, for example, the second quantity of at least one emollient may weigh at least 10 wt. % relative to the weight of the quantity of lip balm, for example at least 15 wt. %, at least 20 wt. %, or the second quantity of at least one emollient may weigh at least 25 wt. % relative to the weight of the quantity of lip balm.

Certain embodiments may provide, for example, a lip balm applicator comprising a lower portion, an upper portion removably connected to at least a part of the lower portion, and a mound of transparent lip balm (for example a mound of transparent lip balm of one of the foregoing the lip balm compositions) in contact with at least one surface of the lower portion, wherein an application surface (for example a surface for application of the lip balm to lips) of the mound of transparent lip balm is exposed upon removal of the upper portion. In certain further embodiments, for example, the lower portion may comprise a support structure contained, at least partially, within the lower portion. In certain further embodiments, for example, the lower portion may have no moving parts. In certain embodiments, for example, the lip balm may have a hardness in the range of 16-24 mm according to the Hardness Test. In certain embodiments, for example, the lip balm may have a transmittance of at least 80% after 24 hours according to the Transmittance Test. In certain embodiments, for example, the lip balm may have a payoff in the range of 0.2-0.6 grams according to the Payoff Test.

Certain embodiments may provide, for example, a lip balm applicator comprising an enclosure and a mound of one of the foregoing the lip balm compositions enclosed and/or concealed in the lip balm applicator. In cumin embodiments, for example, the enclosure may comprise an upper portion, a lower portion removably connected to the upper portion, and a support structure contained within the lower portion (for example the support structure may be integral to the lower portion and/or the lower portion may comprise the support structure).

Certain embodiments may provide, for example, a mound of lip balm having a base to height ratio of less than 2, a transmittance of at least 80% after 24 hours according to the Transmittance Test, a passing score according to the Droop Test, a payoff in the range of 0.2-0.6 grams according to the Payoff Test, and a moisture retention of at least 70 after 15 minutes according to the Moisture Retention Test. In certain further embodiments, for example, the lip balm composition may pass one or more (for example all) of the Appearance Test, the Color Test, Odor Test, and the Texture Test, each of which is described hereunder. In certain embodiments, for example, the mound of lip balm may be self-supporting. In certain further embodiments, for example, the mound of lip balm may be self-supporting at room temperature. In certain embodiments, for example, the mound of lip balm may be clear. In certain embodiments, for example, the lip balm may have a haze value of less than 10% (for example less than 5%, less than 3%, less than 1%, less than 0.5% or less than 0.1%) according to ASTM D1003.

In certain embodiments, for example, the mound of lip balm may be formed from one of the foregoing disclosed lip balm compositions. In certain further embodiments, for example, the mound of lip balm may comprise in the range of 15-25 wt. % ricinus communis (Castor) seed oil, in the range of 11-17 wt. % dicaprylyl carbonate, in the range of 10-15 wt. % decyl isostearate, in the range of 7-13 wt. % butyrospermum parkii (rhea) oil, in the range of 7-13 wt. % pentaerythrityl tetraisostearate, in the range of 2-6 wt. % isostearyl isostearate, in the range of 2-6 wt. % octyldodecanol, in the range of 10-20 wt. % polyimide-3, in the range of 2-10 wt. % polyamide-8, and in the range of 0.25-2 wt. % silica dimethyl silylate. In certain further embodiments, for example, the lip balm composition may comprise about 20 wt. % ricinus communis (Castor) seed oil, about 14 wt. % dicaprylyl carbonate, about 12.75 wt. % decyl isostearate, about 10 wt. % butyrospermum parkii (shea) oil, about 10.7 wt. % pentaerythrityl tetraisostearate, about 4.25 wt. % isostearyl isostearate, about 4 wt. % octyldodecanol, about 15 wt. % polyamide-3, about 5 wt. % polyamide-8, about 1 wt. % silica dimethyl silylate, about 0.1 wt. % cocas nucifera (coconut) oil, about 0.1 wt. % helianthus annuus (sunflower) seed oil, about 0.1 wt. % persea gratissima (avocado) oil, about 1 wt. % flavor FZ9255, and about 2 wt. % sweetness enhancer FZ7492.

Certain embodiments may provide, for example, a lip balm applicator comprising: an upper portion, a lower portion removably connected to the upper portion, (and optionally a support platform attached to, or formed integrally with, the lower portion) and a mound of transparent lip balm in contact with the optional support platform and/or at least one surface of the lower portion. In certain further embodiments, for example, the mound of transparent lip balm may be preservative-free, non-telescoping and non-articulating relative to the lower portion, and may have a clear color according to the Color Test. In certain embodiments, for example, the mound of transparent lip balm may comprise at least one emollient, at least one structuring polymer, and at least one non-silicone silicon compound (for example fumed silica or silica dimethyl silylate). In certain further embodiments, for example, the at least one emollient may comprise greater than 10 wt. % castor oil and in the range of 10-40 wt. % dicaprylyl carbonate. In certain embodiments, for example, the mound of transparent lip balm may be self-supporting at room temperature.

Certain embodiments may provide, for example, a lip balm applicator comprising: an enclosure comprising a lower portion and an upper portion removably connected to at least a part of the lower portion, and a mound of transparent lip balm enclosed within the enclosure. In certain further embodiments, for example, the mound of transparent lip balm may have an application surface extending from and exposed beyond the lower portion, the application surface having an application surface area of greater than 55% of the total surface area of the mound, and a transmittance of at least 93% after 24 hours according to the Transmittance Test. In certain further embodiments, for example, the lower portion may comprise a support structure contained at least partially within the lower portion. In certain embodiments, for example, the mound of transparent lip balm may not contact the upper portion of the enclosure when said upper portion of the enclosure and said lower portion of the enclosure are connected to enclose the lip balm. In certain embodiments, for example, the mound of lip balm may only contact the support structure, exclusive of the rest of the lower portion and the upper portion. In certain further embodiments, for example, the mound of transparent lip balm may have a passing score according to the Droop Test after 1-week at 50° C., and/or a payoff in the range of 0.2-0.6 grams according to the Payoff Test, and/or a moisture retention of at least 70 after 15 minutes according to the Moisture Retention Test.

Certain embodiments may provide, for example, a lip balm applicator comprising an enclosure and a mound of transparent lip balm (for example one of the mounds of lip balm disclosed above) enclosed and/or concealed in or within the lip balm applicator. In certain embodiments, for example, the enclosure may comprise an upper portion, a lower portion removably connected to the upper portion, and a support structure, for holding the lip balm mound, contained at least partially within the lower portion. In certain embodiments, for example, the mound of transparent lip balm may have a transmittance of at least 80% (for example at least 93%) after 24 hours according to the Transmittance Test. In certain further embodiments, for example, the mound of transparent lip balm may have a mound surface, and the mound surface may comprise a support surface contained within the lower portion, and an application surface extending from (for example positioned above) the lower portion. In certain further embodiments, for example, the application surface area may have a surface area of greater than 50% (for example greater than 60%, 66%, 70%, or greater than 80%) of the surface area of the mound of transparent lip balm, wherein the lip balm mound only contacts the lower portion when enclosed within the enclosure. In certain embodiments, for example, the mound of transparent lip balm may not contact the upper portion of the enclosure when said upper portion of the enclosure and said lower portion of the enclosure are connected to enclose the lip balm. In certain embodiments, for example, the mound of lip balm may only contact the support structure, exclusive of the rest of the lower portion and the upper portion.

In certain embodiments, for example, at least a portion of the support surface may be in contact with the support structure. In certain further embodiments, for example, at least a portion of the support surface may be formed around a portion of the support structure.

In certain embodiments, for example, the mound of lip balm may be in fixed relation with the lower portion. In certain embodiments, for example, the support platform and/or the lower portion holding the mound of lip balm may have no moving parts. In certain embodiments, for example, the mound of lip balm may not be articulated in relation to the lower portion. In certain embodiments, for example, the mound of lip balm may be non-telescoping, non-extendable and non-retractable in relation to the lower portion. In certain embodiments, for example, the mound of lip balm may be in fixed relation with the enclosure. In certain embodiments, for example, the mound of lip balm may not be articulated in relation to the enclosure. In certain embodiments, for example, the mound of lip balm may be non-telescoping, non-extendable and non-retractable in relation to the enclosure.

In certain embodiments, for example, the application surface area may comprise more than 50% of the surface area of the mound of transparent lip balm, for example the application surface area may comprise more than 55% of the surface area of the mound of transparent lip balm, more than 60%, more than 65%, more than 66%, more than 67%, more than 68%, more than 69%, more than 70%, more than 75%, more than 80%, or the application surface area may comprise more than 85% of the surface area of the mound of transparent lip balm.

In certain embodiments, for example, the mound of lip balm of the lip balm applicator may have a passing score according to the Droop Test. In certain embodiments, for example, the mound of lip balm of the lip balm applicator may have a payoff in the range of 0.2-0.6 grams according to the Payoff Test. In certain embodiments, for example, the mound of lip balm of the lip balm applicator may have a moisture retention of at least 70 after 15 minutes according to the Moisture Retention Test. In certain embodiments, for example, the lip balm composition may pass one or more (for example all) of the Appearance Test, the Color Test, Odor Test, the Texture Test, and the Packaging Test (described hereunder). In certain embodiments, for example, the lip balm may have a haze value of less than 10% (for example less than 5%, less than 3%, less than 1%, less than 0.5%, or less than 0.1%) according to ASTM D1003.

Certain embodiments may provide, for example, a method comprising removing the upper portion of one of the foregoing lip balm applicators and contacting the application surface to skin.

Certain embodiments may provide, for example, a lip balm applicator comprising an enclosure comprising an upper portion and a lower portion, a support platform within the enclosure, and a mound of transparent lip balm (for example one of the foregoing disclosed mounds of lip balm) in contact with the support platform, the mound configured to optically magnify the support platform as viewed from above the mound, said mound having a transmittance of at least 80% (for example at least 93%) after 24 hours according to the Transmittance Test. In certain further embodiments, for example, the support platform may comprise a hollow (or open) inner hub.

In certain embodiments, for example, the magnification may be effective to magnify an inner portion of the support platform (for example a hollow inner hub) by at least 100% (for example at least 200% or at least 300%). In certain embodiments, for example, the magnification of the support platform may be effective to remove at least 50% (for example at least 75%) of the support platform from the field of view when viewed from directly above the hollow inner hub. In certain embodiments, for example, the magnification of the support platform may be effective to make it appear that the mound of lip balm is supported by less than 50% of the visible support structure (for example less than 40%, less than 25%, or less than 15% when viewed from directly above the hollow inner hub. In certain embodiments, for example, the magnification may be effective to magnify a feature separate from the support platform (for example a mark such as a trademark or other words present below the support platform) when viewed from directly above the hollow inner hub. Certain embodiments may provide, for example, a method to improve product source identification, product marking and/or reduce the likelihood of consumer confusion regarding the source of the lip balm applicator, comprising: positioning a visible trademark below the hollow inner hub of the support structure of the foregoing lip balm applicator, whereby the trademark may be magnified when viewed from above (for example from directly above) the hollow inner hub. Certain embodiments may provide, for example, a method to enhance the lip balm applicator, comprising: positioning a visible design or design feature in or below the inner portion of the support structure, whereby the design or design feature may be magnified when viewed through the mound of transparent lip balm.

In any of the foregoing embodiments, or any other embodiments disclosed herein, the lip balm may be in the form of a stick, for example a stick may be used in place of a mound in any of the foregoing embodiments. In certain embodiments, for example, the stick may have any elongated configuration, such as a cylinder or cuboid (or any shape derived therefrom), for example a cylinder tapered or rounded at one end (such as a cylinder tapered at an application end).

### BRIEF DESCRIPTION OF THE DRAWINGS

Exemplary embodiments will now be described, by way of example only, with reference to the accompanying drawings in which

FIG. 1 is a perspective view of an embodiment of a lip balm applicator.

FIG. 2 is a top view of the lip balm applicator depicted in FIG. 1.

FIG. 3 is a perspective view of an embodiment of a lip balm applicator.

FIG. 4 is a top view of the lip balm applicator depicted in FIG. 3.

FIG. 5 is a perspective view of an embodiment of a lip balm applicator.

FIG. 6 is a top view of the lip balm applicator depicted in FIG. 5.

FIG. 7 is a top view of an embodiment of a lip balm applicator.

FIG. 8 is a perspective view of an embodiment of a lip balm applicator.

FIG. 9 is a top view of the lip balm applicator depicted in FIG. 8.

FIG. 10 is a perspective view of an embodiment of a lip balm applicator.

FIG. 11 is a top view of the lip balm applicator depicted in FIG. 10.

### DETAILED DESCRIPTION

The present disclosure is described in further detail with reference to one or more embodiments, some examples of which are illustrated in the accompanying drawings. The examples and embodiments are provided by way of explanation and are not to be taken as limiting to the scope of the disclosure. Furthermore, features illustrated or described as part of one embodiment may be used by themselves to provide other embodiments and features illustrated or described as part of one embodiment may be used with one or more other embodiments to provide further embodiments. The present disclosure covers these variations and embodiments as well as other variations and/or modifications.

In certain embodiments, for example, the lip balm may have substantial transparency and may be hard enough to be suitable for commercial use with a lip balm container that exposes a substantial percentage of the surface area of the lip balm for application (for example, a mounded lip balm or a hemispherical or hemi-egg shaped mounded lip balm). In certain embodiments, for example, the lip balm may be mounded on a support platform of the container. In certain further embodiments, the lip balm may lock onto support surfaces on the support platform and/or a lower portion of the container. In certain embodiments, for example, the lip balm may have sufficient hardness and stiffness to effectively grasp the support platform without becoming dislodged and/or breaking off during use. In certain embodiments, for example, the lip balm may differ from lip balms in tubes, jars, or tins because a substantial portion of the surface area of the lip balm may be exposed for application when the lip balm applicator is opened without any support at its periphery. Moreover, in certain embodiments, for example, unlike lip balms in tubes, jars, or tins, the exposable surface area of the lip balm may not have any periphery support even when the lip balm applicator is closed. Accordingly, in such embodiments without periphery support, the exposed lip balm may resist droop. Furthermore, to the extent the container may have a threaded top portion, the exposed lip balm must adequately resist deforming under torsional forces caused by opening and closing the container. In certain embodiments, for example, the lip balm may be transparent after using a “hot pour” process to form the mound.

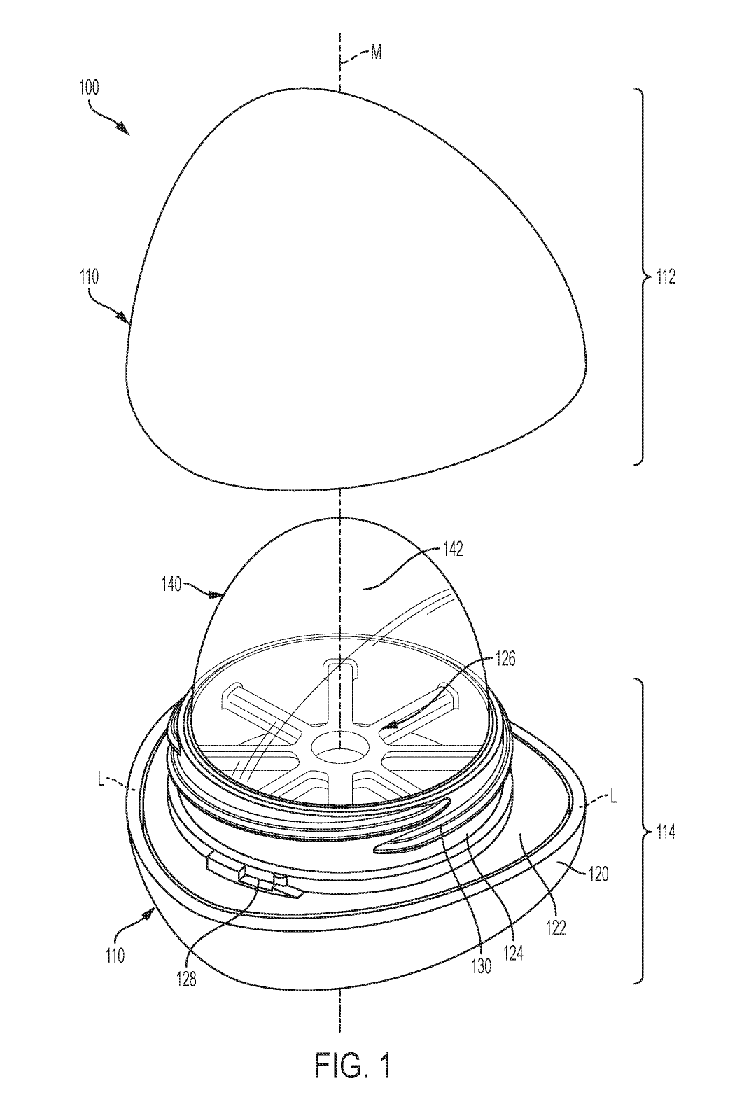

Depicted in FIG. 1 is an embodiment of a lip balm applicator 100. The applicator 100 comprises a enclosure 110 having the lip balm composition enclosed therein. The enclosure 110 in this embodiment has a particular shape. The present invention is not limited in this regard, as the enclosure 110 may be substantially cylindrical with rounded surfaces, egg-shaped, ovate, pyramidal, spherical, cubic, box-like, or many other shapes. The enclosure 110 comprises an upper portion 112 and a lower portion 114, the portions being separable from each other along a line L that extends substantially circumferentially about the enclosure perpendicular to a major axis M extending longitudinally through the enclosure.

The lower portion 114 comprises an outer shell 120, a generally horizontal shoulder 122 (relative to axis M), a generally vertical neck 124, and a support platform 126. The neck 124 further comprises threads 130 and at least one locking member 128 whereby the lower portion 114 may be connected, e.g., interengaged and reversibly connected (i.e., fit together frictionally) with the upper portion 112.

Connected to the lower portion 114 is a mound of transparent lip balm 140 mounted on the support platform 126. The mound of transparent lip balm 140 is exposed upon removal of the upper portion 112 from the lower portion 114. The lip balm 140 in this embodiment comprises a quantity of lip balm material formed or otherwise configured to have a mound shape, for example a partial (or hemi-) oval, cubical, pyramidal, spherical shape or any number of other shapes of mounds, that provide an exposed surface of application extending from the lower portion, for example a curved application surface 142. The mound shape and surface 142 allow the lip balm material to extend above the upper edge of the lower portion 114, thereby facilitating the application of the lip balm to a user's lips without having to move the lip balm, relative to the lower portion, for example without having to extend or retract the lip balm, relative to the container, by for example some mechanical means.

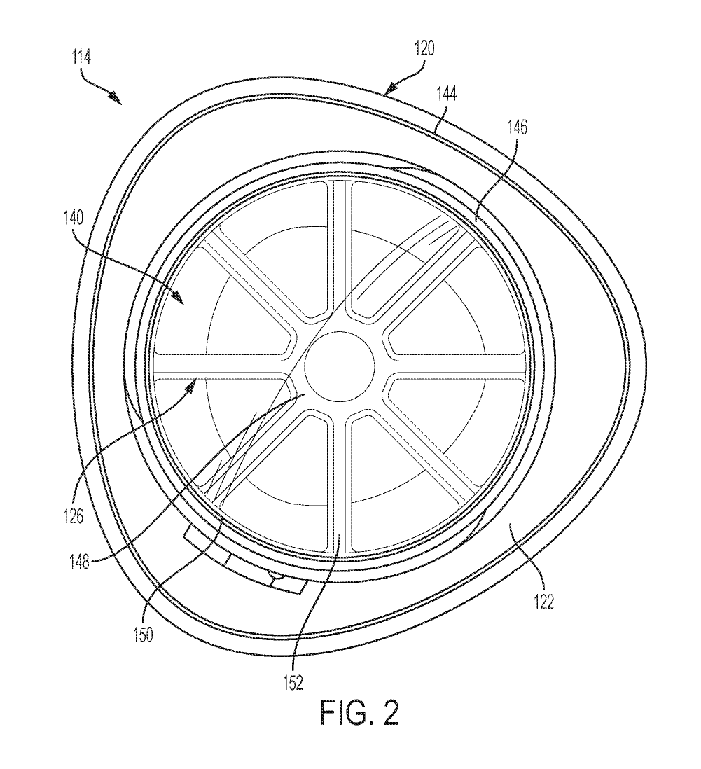

FIG. 2 is a top view of the lower portion 114 of FIG. 1. The generally horizontal shoulder 122 may be integrally connected to the interior 144 of the outer shell 120, and the support platform 126 may be integrally connected to the interior of the neck 146.

The support platform 126, in this embodiment, is a hub-and-spoke assembly comprising a hollow inner hub 148, an outer rim 150, and a plurality of spokes 152 that extend from the inner hub 148 and terminate at the outer rim 150. The diameter and thickness of the inner hub 148, the outer rim 150 and the number and thickness of the spokes 152 may vary and may be designed to provide a suitable area on which the lip balm 140 can be supported. The spokes 152 extend from the inner hub 148 at equal angles and connect with an inner surface of the outer rim 150.

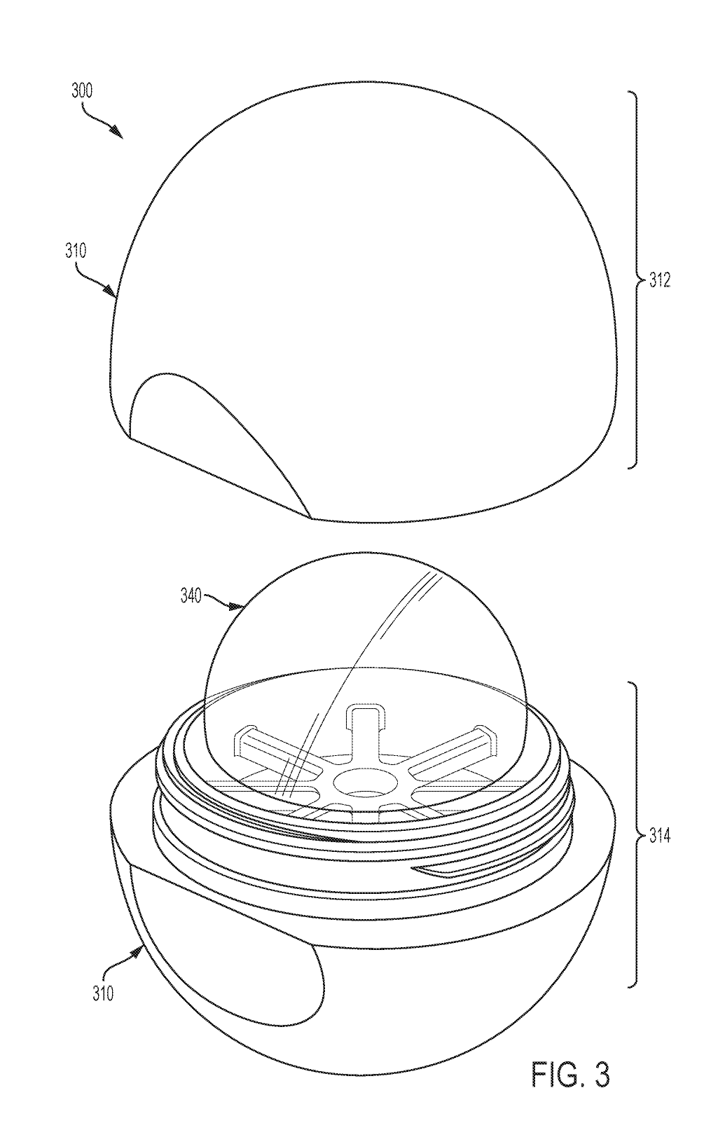

Depicted in FIG. 3 is an embodiment of a lip balm applicator 300. The applicator 300 comprises a enclosure 310 having a mound of transparent lip balm 340 enclosed therein. The enclosure 310 in this embodiment forms a substantially egg-shaped enclosure and the lip balm 340 is substantially hemispherical. The enclosure 310 comprises an upper portion 312 and a lower portion 314, the portions being separable from each other.

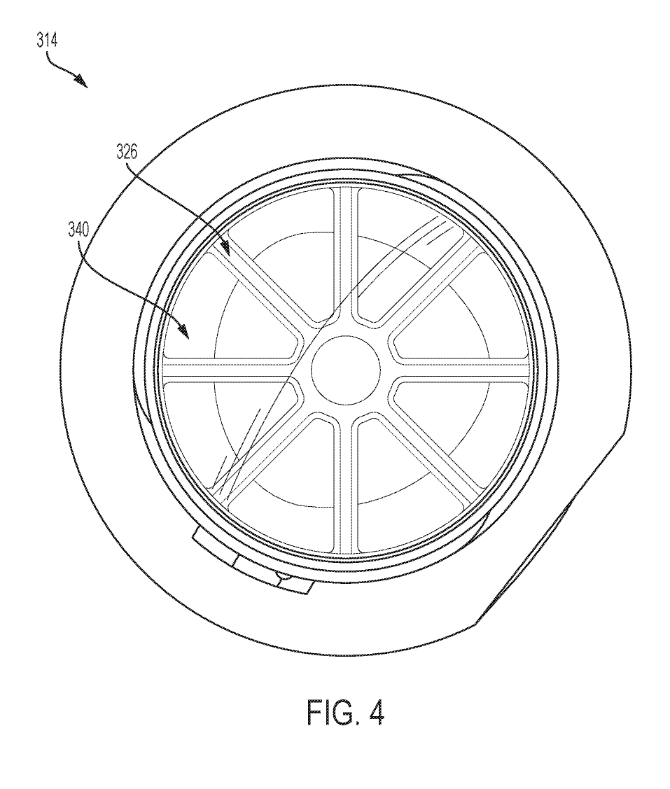

FIG. 4 is a top view of the lower portion 314 of FIG. 3. The support platform 326 supports the lip balm 340.

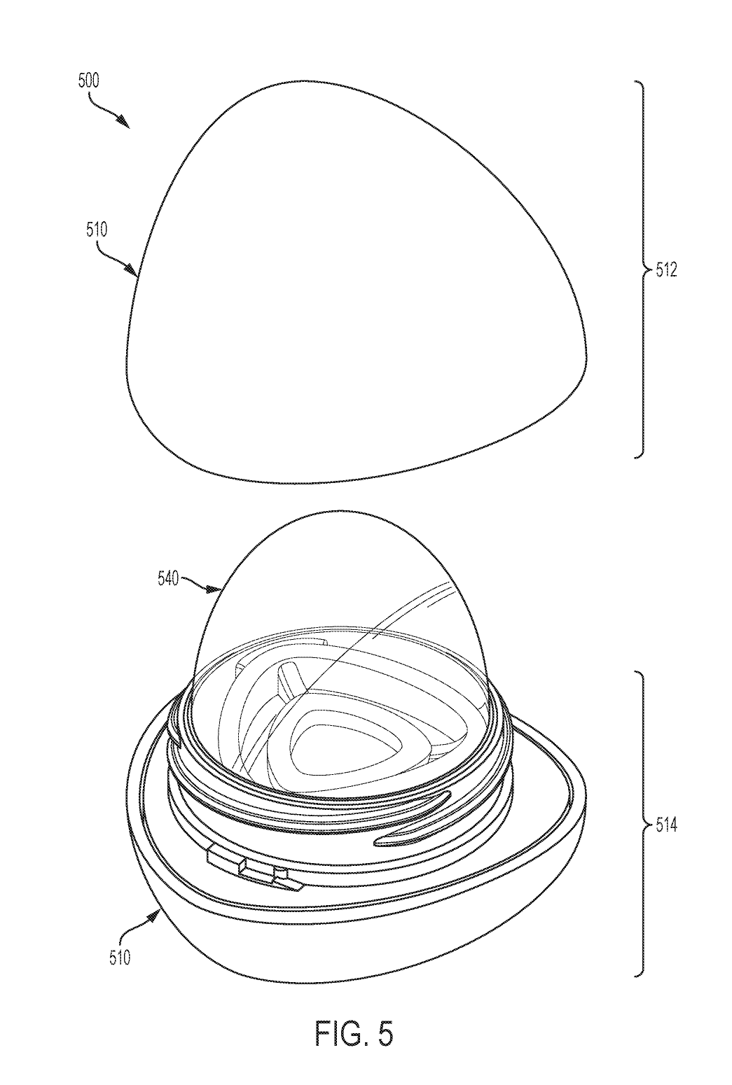

Depicted in FIG. 5 is an embodiment of a lip balm applicator 500. The applicator 500 comprises a enclosure 510 having a mound of transparent lip balm 540 enclosed therein. The enclosure 510 in this embodiment forms a substantially rounded enclosure and the lip balm 540 has substantially oval shape. The enclosure 510 comprises an upper portion 512 and a lower portion 514, the portions being separable from each other.

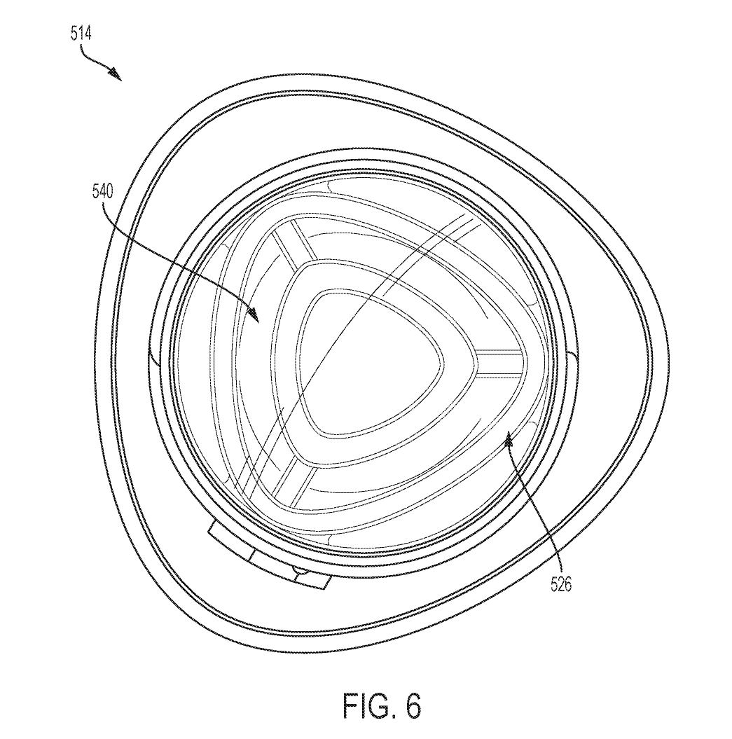

FIG. 6 is a top view of the lower portion 514 of FIG. 5. The support platform 526 supports the lip balm 540.

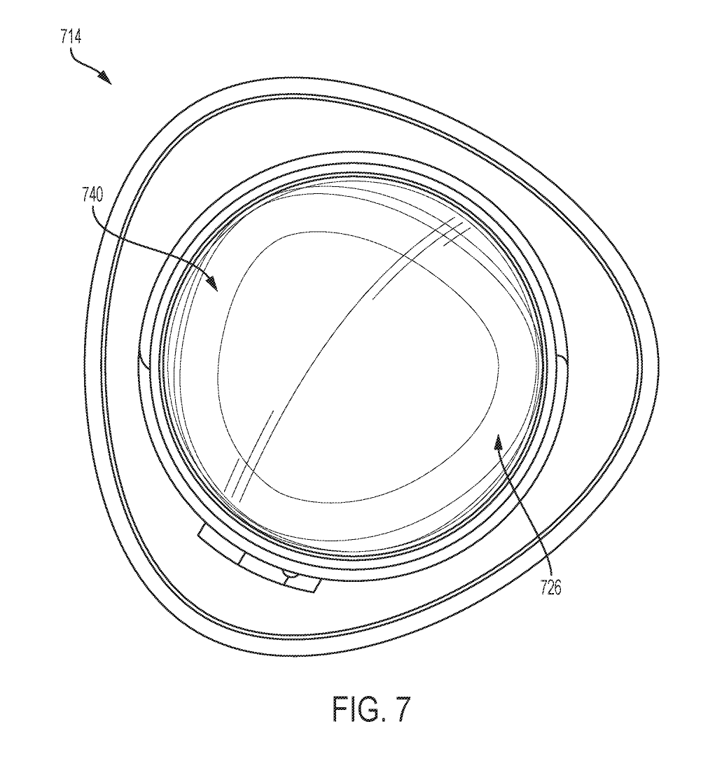

FIG. 7 is a top view of a lower portion 714 of a lip balm enclosure. A support platform 726 supports a mound of transparent lip balm 740.

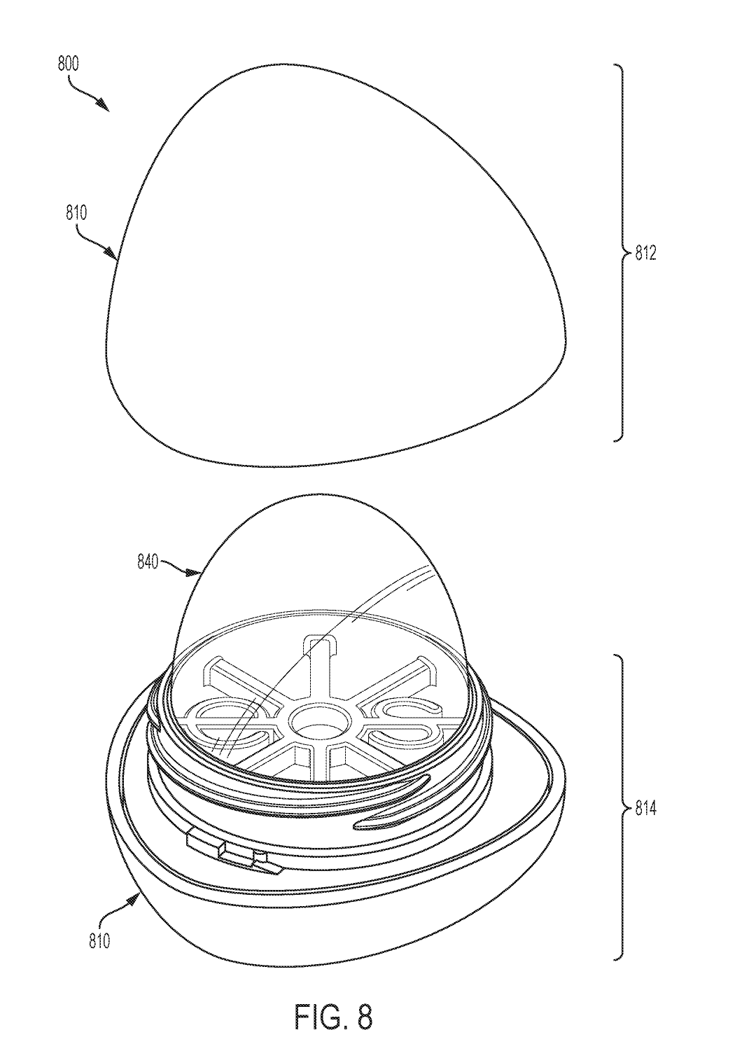

Depicted in FIG. 8 is an embodiment of a lip balm applicator 800. The applicator 800 comprises a enclosure 810 having a mound of transparent lip balm 840 enclosed therein. The enclosure 810 in this embodiment forms a substantially rounded enclosure and the lip balm 840 has substantially oval shape. The enclosure 810 comprises an upper portion 812 and a lower portion 814, the portions being separable from each other.

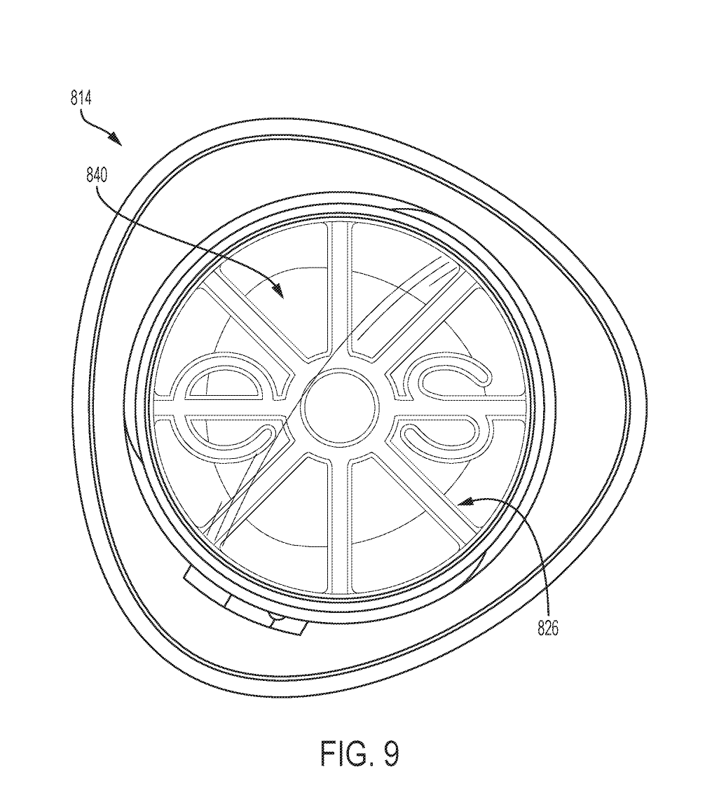

FIG. 9 is a top view of the lower portion 814 of FIG. 8. The support platform 826 supports the lip balm 840.

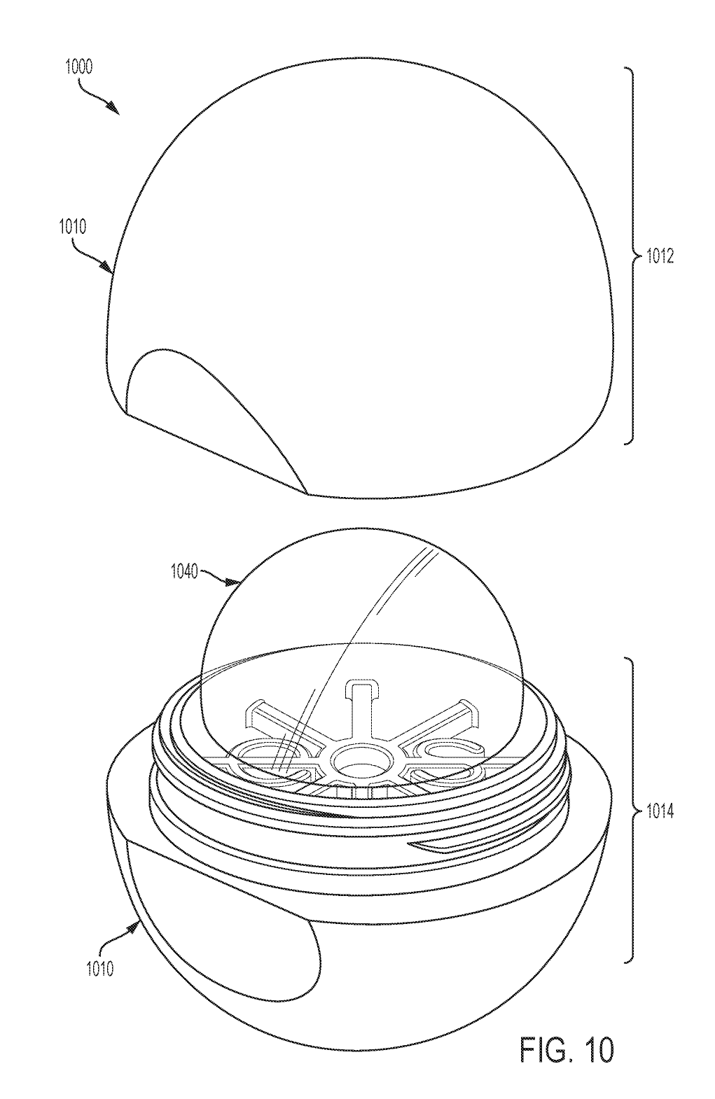

Depicted in FIG. 10 is an embodiment of a lip balm applicator 1000. The applicator 1000 comprises a enclosure 1010 having a mound of transparent lip balm 1040 enclosed therein. The enclosure 1010 in this embodiment forms a substantially egg-shaped enclosure and the lip balm 1040 is substantially hemispherical. The enclosure 1010 comprises an upper portion 1012 and a lower portion 1014, the portions being separable from each other.

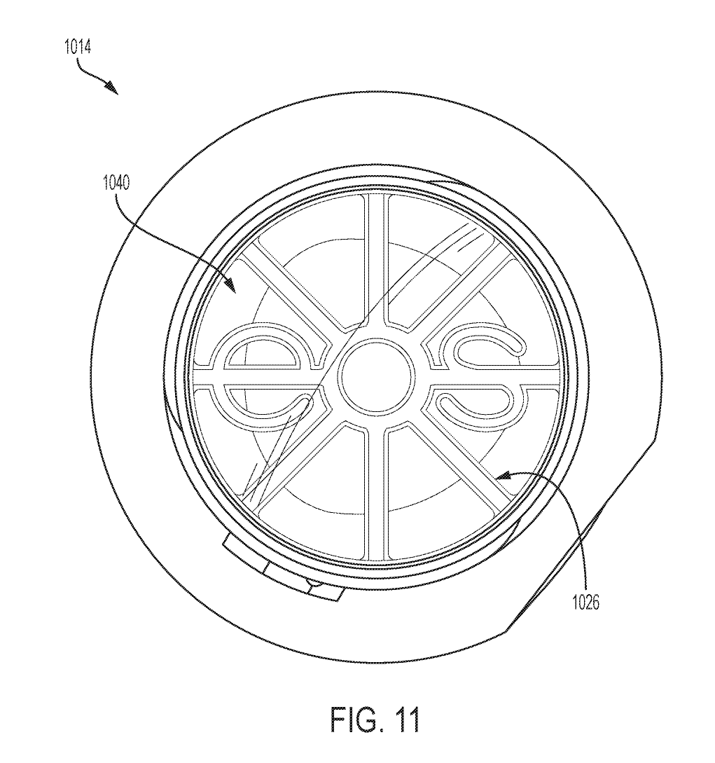

FIG. 11 is a top view of the lower portion 1014 of FIG. 10. The support platform 1026 supports the lip balm 1040.

Certain embodiments may provide, for example, a lip product such as lip stick, lip balm, and/or lip gloss. In certain further embodiments, for example, the lip product may be a mound. In certain embodiments, for example, the lip product may be any elongated shape, for example a stick such as a cylinder or a cuboid. In certain further embodiments, for example, the elongated shape may be tapered at one or both ends, for example the elongated shape may be tapered on an application end.

In certain embodiments, for example, the lip product may include a combination of one or more structuring agents, emollients, moisturizers, oils, essential oils, lipids, thickening agents, stability agents, shine enhancers, natural flavors, artificial flavors, odorants, sunscreen agents (for example UV blocking agents), cosmetic ingredients, and/or antioxidants. The foregoing categories are not mutually exclusive (for example an oil might also be an emollient). A wide variety of such ingredients may be used. Examples of some ingredients are provided herein.

Structuring Agents: One or more structuring agents may be used, for example, to supply a required level of stiffness and hardness to a lip balm composition. In certain embodiments, for example, one or more structuring agents may be employed, in combination with other ingredients, to form a self-supporting lip balm composition. In certain further embodiments, for example, the self-supporting lip balm composition may pass the Droop Test. In certain embodiments, for example, the one or more structuring agents may comprise polyamides including polyamide-8 and polyamide-3, wax, silica, stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, or the polyethylene glycol ether of cetyl alcohol having an average of about 1 to about 5 ethylene oxide units, or a combination of two of more thereof. In certain embodiments, for example, the one or more structuring agents (for example one or more polyamides) may contain in the range of 1-5 wt. %, 5-10 wt. %, 10-15 wt. %, 15-20 wt. %, 20-25 wt. %, 25-30 wt. %, 30-35 wt. %, or 35-40 wt. % of the one or more structuring agents, relative to the total weight of the lip balm composition. In certain embodiments, for example, the lip balm composition may contain in the range of 10-30 wt. % of the one or more structuring agents (for example one or more polyamides) relative to the total weight of the lip balm composition, for example in the range of 15-25 wt. %, or the lip balm composition may contain in the range of 18-22 wt. % of the one or more structuring agents relative to the total weight of the lip balm composition. In certain embodiments, for example, the lip balm composition may contain 1 wt. % of the one or more structuring agents (for example one or more polyamides), relative to the total weight of the lip balm composition, for example 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, 30 wt. %, 31 wt. %, 32 wt. %, 33 wt. %, 34 wt. %, 35 wt. %, 36 wt. %, 37 wt. %, 38 wt. %, 39 wt. %, or the lip balm composition may contain 40 wt. % of the one or more structuring agents, relative to the total weight of the lip balm composition. In certain embodiments, for example, the lip balm composition may contain less than 50 wt. % of the one or more structuring agents (for example one or more polyamides), relative to the total weight of the lip balm composition, for example less than 40 wt. %, less than 30 wt. %, less than 25 wt. %, less than 20 wt. %, less than 15 wt. %, or the lip balm composition may contain less than 10 wt. % of the one or more structuring agents, relative to the total weight of the lip balm composition.

In certain embodiments, for example, the lip balm composition may contain 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, or 30 wt. % polyamide-3, relative to the total weight of the lip balm composition. In certain embodiments, for example, the lip balm contain may contain in the range of 1-5 wt. %, 5-10 wt. %, 10-15 wt. %, 15-20 wt. %, 20-25 wt. %, or 25-30 wt. % polyamide-3, relative to the total weight of the lip balm composition. In certain embodiments, for example, the lip balm composition may contain in the range of 10-20 wt. % polyamide-3 relative to the total weight of the lip balm composition, for example in the range of 12-18 wt. %, or the lip balm composition may contain in the range of 14-16 wt. % polyamide-3 relative to the total weight of the lip balm composition.

In certain embodiments, for example, the lip balm composition may contain 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, or 30 wt. % polyamide-8, relative to the total weight of the lip balm composition. In certain embodiments, for example, the lip balm contain may contain in the range of 1-5 wt. %, 5-10 wt. %, 10-15 wt. %, 15-20 wt. %, 20-25 wt. %, or 25-30 wt. % polyamide-8, relative to the total weight of the lip balm composition. In certain embodiments, for example, the lip balm composition may contain in the range of 2-10 wt. % polyamide-8, for example in the range of 3-7 wt. %, or the lip balm composition may contain in the range of 4-6 wt. % polyamide-8, relative to the total weight of the lip balm composition.

In certain embodiments, the lip balm composition may contain in the range of 15-25 wt. % polyamide-3 and 2-10 wt. % polyamide-8, relative to the total weight of the lip balm composition, for example the lip balm composition may contain in the range of 18-22 wt. % polyamide-3 and 4-7 wt. % polyamide-8, relative to the total weight of the lip balm composition.

In certain embodiments, for example, the lip balm composition may comprise one or more emollients used, for example, as a friction-reducing lubricant on the skin while helping to moisturize the skin (i.e., keep the skin moist and supple) by softening and smoothing the scales of the skin while providing a layer of protection that helps prevent moisture (water) loss. In certain embodiments, for example, the one or more emollients may be an amino acid, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, xylitol, acetylated lanolin, acetylated lanolin alcohol, alanine, algae extract, aloe barbadensis, aloe-barbadensis extract, aloe barbadensis gel, althea officinalis extract, apricot (prunus armeniaca) kernel oil, arginine, arginine aspartate, arnica montana extract, aspartic acid, avocado (persea gratissima) oil, barrier sphingolipids, butyl alcohol, beeswax, behenyl alcohol, beta-sitosterol, birch (betula alba) bark extract, borage (borago officinalis) extract, butcherbroom (ruscus aculeatus) extract, butylene glycol, calendula officinalis extract, calendula officinalis oil, candelilla (euphorbia cerifera) wax, canola oil, caprylicicapric triglyceride, cardamon (elettaria cardamomum) oil, carnauba (copernicia cerifera) wax, carrot (daucus carota sativa) oil, castor (ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (anthemis nobilis) oil, cholesterol, cholesterol esters, cholesterol hydroxystearate, citric acid, clary (salvia sclarea) oil, cocoa (theobroma cacao) butter, coco-caprylate/caprate, coconut (cocos nucifera) oil, collagen, collagen amino acids, corn (zea mays) oil, fatty acids, decyl isostearate, decyl oleate, dicaprylyl carbonate, dimethicone copolyol, dimethiconol, dioctyl adipate, dioctyl succinate, dipentaerythrityl hexacaprylate/hexacaprate, DNA, erythritol, ethoxydiglycol, ethyl linoleate, eucalyptus globulus oil, evening primrose (oenothera biennis) oil, fatty acids, geranium maculatum oil, glucosamine, glucose glutamate, glutamic acid, giycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (vitis vinifera) seed oil, hazel (corylus americana) nut oil, hazel (corylus avellana) nut oil, hexylene glycol, hyaluronic acid, hybrid safflower (carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (jasminum officinale) oil, jojoba (buxus chinensis) oil, kelp, kukui (aleurites moluccana) nut oil, lactamide MEA, laneth-16, laneth-10 acetate, lanolin, lanolin acid, lanolin alcohol, lanolin oil, lanolin wax, lavender (lavandula angustifolia) oil, lecithin, lemon (citrus medica litnonutn) oil, linoleic acid, linolenic acid, macadamia ternifolia nut oil, maltitol, matricaria (chamomilla recutita) oil, methyl glucose sesquistearate, methylsilanol PCA, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (olea europaea) oil, orange (citrus aurantium dulcis) oil, palm (elaeis guineensis) oil, palmitic acid, pantethine, panthenol, panthenyl ethyl ether, paraffin, PCA, peach (prunes persica) kernel oil, peanut (arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG40 sorbitan peroleate, PEG-5 soy sterol, PEG-10 soy sterol, PEG-2 stearate, PEG-8 stearate, PEG-20 stearate, PEG-32 stearate, PEG-40 stearate, PEG-50 stearate, PEG-100 stearate, PEG-150 stearate, pentadecalactone, peppermint (mentha piperita) oil, petrolatum, phospholipids, polyamine sugar condensate, polyglyceryl-3 diisostearate, polyquaternium-24, polysorbate 20, polysorbate 40, polysorbate 60, polysorbate 80, polysorbate 85, potassium myristate, potassium palmitate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, retinol, retinyl palmitate, rice (oryza sativa) bran oil, RNA, rosemary (rostnarinus officinalis) oil, rose oil, safflower (carthamus tinctorius) oil, sage (salvia officinalis) oil, sandalwood (santalum album) oil, serine, serum protein, sesame (sesamum indicum) oil, shea butter (butyrospermum parkii), silk powder, sodium chondroitin sulfate, sodium hyaluronate, sodium lactate, sodium palmitate, sodium PCA, sodium polyglutamate, soluble collagen, sorbitan laurate, sorbitan oleate, sorbitan palmitate, sorbitan sesquioleate, sorbitan stearate, sorbitol, soybean (glycine soja) oil, sphingolipids, squalene, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sweet almond (prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (triticum vulgare) germ oil, ylang ylang (cananga odorata) oil, decyl isostearate, isostearyl isostearate, octyldodecanol, pentaerythrityl tetraisostearate, isostearyl isostearate, isotearyl alcohol, PPG-3 Benzyl Ether Ethylhexanoate, cetearyl ethylhexanoate, or a combination of two or more (for example a combination of 2, 3, 4, 5, 6, or 7 different emollients) thereof. In certain embodiments, for example, the lip balm composition may comprise in the range of 1-5 wt. % of the one or more emollients, relative to the total weight of the lip balm composition, for example in the range of 5-10 wt. %, in the range of 10-15 wt. %, in the range of 15-20 wt. %, in the range of 20-25 wt. %, in the range of 25-30 wt. %, in the range of 30-35 wt. %, in the range of 35-40 wt. %, in the range of 40-45 wt. %, in the range of 45-50 wt. %, in the range of 55-60 wt. %, in the range of 65-70 wt. %, in the range of 60-80 wt. %, in the range of 60-85 wt. %, in the range of 75-80 wt. %, in the range of 80-85 wt. %, or the lip balm may comprise in the range of 85-90 wt. % of the one or more emollients, relative to the total weight of the lip balm composition. In certain embodiments, for example, the lip balm composition may comprise 1 wt. % of the one or more emollients, relative to the total weight of the lip balm composition, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, 30 wt. %, 31 wt. %, 32 wt. %, 33 wt. %, 34 wt. %, 35 wt. %, 36 wt. %, 37 wt. %, 38 wt. %, 39 wt. %, 40 wt. %, 41 wt. %, 42 wt. %, 43 wt. %, 44 wt. %, 45 wt. %, 46 wt. %, 47 wt. %, 48 wt. %, 49 wt. %, 50 wt. %, 51 wt. %, 52 wt. %, 53 wt. %, 54 wt. %, 55 wt. %, 56 wt. %, 57 wt. %, 58 wt. %, 59 wt. %, 60 wt. %, 61 wt. %, 62 wt. %, 63 wt. %, 64 wt. %, 85 wt. %, 66 wt. %, 67 wt. %, 68 wt. %, 69 wt. %, 70 wt. %, 71 wt. %, 72 wt. %, 73 wt. %, 74 wt. %, 75 wt. %, 76 wt. %, 77 wt. %, 78 wt. %, 79 wt. %, 80 wt. %, 81 wt. %, 82 wt. %, 83 wt. %, 84 wt. %, 85 wt. %, 86 wt. %, 87 wt. %, 88 wt. %, 89 wt. %, or the lip balm composition may comprise 90 wt. % of the one or more emollients, relative to the total weight of the lip balm composition. In certain embodiments, for example, the lip balm composition may comprise less than 75 wt. % of the one or more emollients, relative to the total weight of the lip balm composition, for example less than 65 wt. %, less than 60 wt. %, less than 50 wt %, or the lip balm composition may comprise less than 45 wt. % of the one or more emollients, relative to the total weight of the lip balm composition.

In certain embodiments, for example, the lip balm composition may contain 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, or 30 wt. % ricinus communis (Castor) seed oil. In certain embodiments, for example, the lip balm composition may contain in the range of 1-5 wt. %, 5-10 wt. %, 10-15 wt. %, 15-20 wt. %, 20-25 wt. %, or 25-30 wt. % ricinus communis (Castor) seed oil. In certain embodiments, for example, the lip balm composition may comprise in the range of 10-30 wt. % ricinus communis (Castor) seed oil, for example in the range of 15-25 wt. %, or the lip balm composition may comprise in the range of 18-22 wt. % ricinus communis (Castor) seed oil. In certain embodiments, for example, the lip balm composition may comprise less than 35 wt. % ricinus communis (Castor) seed oil, for example less than 25 wt. %, less than 20 wt. %, less than 15 wt. %, or the lip balm composition may comprise less than 10 wt. % ricinus communis (Castor) seed oil.

In certain embodiments, for example, the lip balm composition may contain 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, or 30 wt. %, 31 wt. %, 42 wt. %, 33 wt. %, 34 wt. %, 35 wt. %, 36 wt. %, 37 wt. %, 38 wt. %, 39 wt. %, or 40 wt. %, 31 wt. %, 42 wt. %, 43 wt. %, 44 wt. %, 45 wt. %, 46 wt. %, 47 wt. %, 48 wt. %, 49 wt. %, or 50 wt. % dicaprylyl carbonate. In certain embodiments, for example, the lip balm composition may contain in the range of 1-5 wt. %, 5-10 wt. %, 10-15 wt. %, 15-20 wt. %, 20-25 wt. %, 25-30 wt. %, 30-35 wt. %, 35-40 wt. %, 40-45 wt. %, 45-50 wt. %, or 50-55 wt. % dicaprylyl carbonate. In certain embodiments, for example, the lip balm composition may comprise in the range of 10-60 wt. % dicaprylyl carbonate, for example in the range of 10-30 wt. %, 12-16 wt. %, 40-50 wt. %, or the lip balm composition may comprise in the range of 45-50 wt. % dicaprylyl carbonate. In certain embodiments, for example, the lip balm composition may comprise less than 35 wt. % dicaprylyl carbonate, for example less than 30 wt. %, less than 25 wt. %, less than 20 wt. %, less than 15 wt. %, or the lip balm composition may comprise less than 10 wt. % dicaprylyl carbonate.

In certain embodiments, for example, the lip balm composition may contain 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, or 30 wt. % octyldodecanol. In certain embodiments, for example, the lip balm composition may contain in the range of 1-5 wt. %, 5-10 wt. %, 10-15 wt. %, 15-20 wt. %, 20-25 wt. %, or 25-30 wt. % octyldodecanol. In certain embodiments, for example, the lip balm composition may contain in the range of 0-10 wt. % octyldodecanol, for example in the range of 2-6 wt. %, or the lip balm composition may comprise in the range of 3-5 wt. % octyldodecanol. In certain embodiments, for example, the lip balm composition may comprise less than 25 wt. % octyldodecanol, for example less than 15 wt. %, less than 10 wt. %, less than 5 wt %, or the lip balm composition may comprise less than 2 wt. % octyldodecanol.

In certain embodiments, for example, the lip balm composition may contain 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, or 30 wt. % octyldodecyl/PPG-3 myristyl ether dimer dilinoleate. In certain embodiments, for example, the lip balm composition may contain in the range of 1-5 wt. %, 5-10 wt. %, 10-15wt. %, 15-20 wt. %, 20-25 wt. %, or 25-30 wt. % octyldodecyl/PPG-3 myristyl ether dimer dilinoleate. In certain embodiments, for example, the lip balm composition may comprise less than 35 wt. % octyldodecyl/PPG-3 myristyl ether dimer dilinoleate, for example less than 25 wt. %, less than 20 wt. %, less than 15 wt %, or the lip balm composition may comprise less than 10 wt. % octyldodecyl/PPG-3 myristyl ether dimer dilinoleate.

In certain embodiments, for example, the lip balm composition may contain 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, or 30 wt. % isostearyl isostearate. In certain embodiments, for example, the lip balm composition may contain in the range of 1-5 wt. %, 5-10 wt. %, 10-15 wt. %, 15-20 wt. %, 20-25 wt. %, or 25-30 wt. % isostearyl isostearate. In certain embodiments, for example, the lip balm composition may contain in the range of 2-20 wt. % isostearyl isostearate, for example in the range of 2-6 wt. %, 3-5 wt. %, 8-12 wt. %, or the lip balm composition may comprise in the range of 9-11 wt. % isostearyl isostearate. In certain embodiments, for example, the lip balm composition may comprise less than 35 wt. % isostearyl isostearate, for example less than 25 wt. %, less than 20 wt. %, less than 15 wt %, or the lip balm composition may comprise less than 10 wt. % isostearyl isostearate.

In certain embodiments, for example, the lip balm composition may contain 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, or 30 wt. % decyl isostearate. In certain embodiments, for example, the lip balm composition may contain in the range of 1-5 wt. %, 5-10 wt. %, 10-15 wt. %, 15-20 wt. %, 20-25 wt. %, or 25-30 wt. % decyl isostearate. In certain embodiments, for example, the lip balm composition may contain in the range of 2-20 wt. % decyl isostearate, for example in the range of 7-15 wt. %, 10-14 wt. %, or the lip balm composition may comprise in the range of 11-13 wt. % decyl isostearate. In certain embodiments, for example, the lip balm composition may comprise less than 35 wt. % decyl isostearate, for example less than 25 wt. %, less than 20 wt. %, less than 15 wt %, or the lip balm composition may comprise less than 10 wt. % decyl isostearate.

In certain embodiments, for example, the lip balm composition may contain 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, or 30 wt. % butyrospermum parkii (shea) oil. In certain embodiments, for example, the lip balm composition may contain in the range of 1-5 wt. %, 5-10 wt. %, 10-15 wt. %, 15-20 wt. %, 20-25 wt. %, or 25-30 wt. % butyrospermurn parkii (shea) oil. In certain embodiments, for example, the lip balm composition may contain in the range of 2-20 wt. % butyrospermum parkii (shea) oil, for example in the range of 5-15 wt. %, 8-12 wt. %, or the lip balm composition may comprise in the range of 9-11 wt. % butyrospermum parkii (shea) oil. In certain embodiments, for example, the lip balm composition may comprise less than 35 wt. % butyrospermum parkii (shea) oil, for example less than 25 wt. %, less than 20 wt. %, less than 15 wt %, or the lip balm composition may comprise less than 10 wt. % butyrospermum parkii (shea) oil.

In certain embodiments, for example, the lip balm composition may contain 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, or 30 wt. % pentaerythrityl tetraisostearate. In certain embodiments, for example, the lip balm composition may contain in the range of 1-5 wt. %, 5-10 wt. %, 10-15 wt. %, 15-20 wt. %, 20-25 wt. %, or 25-30 wt. % pentaerythrityl tetraisostearate. In certain embodiments, for example, the lip balm composition may contain in the range of 2-20 wt. % pentaerythrityl tetraisostearate, for example in the range of 5-15 wt. %, 8-12 wt. %, or the lip balm composition may comprise in the range of 9-11 wt. % pentaerythrityl tetraisostearate. In certain embodiments, for example, the lip balm composition may comprise less than 35 wt. % pentaerythrityl tetraisostearate, for example less than 25 wt. %, less than 20 wt. %, less than 15 wt %, or the lip balm composition may comprise less than 10 wt. % pentaerythrityl tetraisostearate.

In certain embodiments, for example, the lip balm composition may contain 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, or 30 wt. % cocos nucifera (coconut) oil. In certain embodiments, for example, the lip balm composition may contain in the range of 0.1-0.5 wt. %, 0.5-1 wt. %, 1-5 wt. %, 5-10 wt. %, 10-15 wt. %, 15-20 wt. %, 20-25 wt. %, or 25-30 wt. % cocos nucifera (coconut) oil. In certain embodiments, for example, the lip balm composition may comprise in the range of 0.0-1 wt. % cocos nucifera (coconut) oil, for example in the range of 0.05-0.25 wt. %, or the lip balm composition may comprise in the range of 0.075-0.125 wt. % cocos nucifera (coconut) oil.

In certain embodiments, for example, the lip balm composition may contain 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, or 30 wt. %, helianthus annuus (sunflower) seed oil. In certain embodiments, for example, the lip balm composition may contain in the range of 0.1-0.5 wt. %, 0.5-1 wt. %, 1-5 wt. %, 5-10 wt. %, 10-15 wt. %, 15-20 wt. %, 20-25 wt. %, or 25-30 wt. % helianthus annuus (sunflower) seed oil. In certain embodiments, for example, the lip balm composition may comprise in the range of 0.0-1 wt. % helianthus annuus (sunflower) seed oil, for example in the range of 0.05-0.25 wt. %, or the lip balm composition may comprise in the range of 0.075-0.125 wt. % helianthus annuus (sunflower) seed oil.

In certain embodiments, for example, the lip balm composition may contain 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, or 30 wt. % aloe barbadensis leaf juice. In certain embodiments, for example, the lip balm composition may contain in the range of 0.1-0.5 wt. %, 0.5-1 wt. %, 1-5 wt. %, 5-10 wt. %, 10-15 wt. %, 15-20 wt. %, 20-25 wt. %, or 25-30 wt. % aloe barbadensis leaf juice. In certain embodiments, for example, the lip balm composition may comprise in the range of 0.0-1 wt. % aloe barbadensis leaf juice, for example in the range of 0.05-0.25 wt. %, or the lip balm composition may comprise in the range of 0.075-0.125 wt. % aloe barbadensis leaf juice.

In certain embodiments, for example, the lip balm composition may contain 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, or 30 wt. % avacado oil. In certain embodiments, for example, the lip balm composition may contain in the range of 0.1-0.5 wt. %, 0.5-1 wt. % 1-5 wt. %, 5-10 wt. %, 10-15 wt. %, 15-20 wt. %, 20-25 wt. %, or 25-30 wt. % avacado oil. In certain embodiments, for example, the lip balm composition may comprise in the range of 0.0-1 wt. % avocado oil, for example in the range of 0.05-0.25 wt. %, or the lip balm composition may comprise in the range of 0.075-0.125 wt. % avocado oil.

In certain embodiments, for example, the lip balm composition may contain 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, or 30 wt. % PPG-3 benzyl ether myristate. In certain embodiments, for example, the lip balm composition may contain in the range of 1-5 wt. %, 5-10 wt. %, 10-15 wt. %, 15-20 wt. %, 20-25 wt. %, or 25-30 wt. % PPG-3 benzyl ether myristate.

In certain embodiments, for example, the lip balm composition may contain 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, or 30 wt. % isostearyl alcohol. In certain embodiments, for example, the lip balm composition may contain in the range of 1-5 wt. %, 5-10 wt. %, 10-15 wt. %, 15-20 wt. %, 20-25 wt. %, or 25-30 wt. % isostearyl alcohol.

In certain embodiments, for example, the lip balm composition may contain 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, or 30 wt. % PPG--3 benzyl ether ethylhexanoate. In certain embodiments, for example, the lip balm composition may contain in the range of 1-5 wt. %, 5-10 wt. %, 10-15 wt. %, 15-20 wt. %, 20-25 wt. %, or 25-30 wt. % PPG-3 benzyl ether ethylhexanoate.

In certain embodiments, for example, the lip balm composition may contain 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, or 30 wt. % cetearyl ethylhexanoate. In certain embodiments, for example, the lip balm composition may contain in the range of 1-5 wt. %, 5-10 wt. %, 10-15 wt. %, 15-20 wt. %, 20-25 wt. %, or 25-30 wt. % cetearyl ethylhexanoate.

Cosmetic Ingredients: In certain embodiments, for example, the lip balm composition may comprise one or more of the cosmetic ingredients disclosed in the CTFA International Cosmetic Ingredient Dictionary and Handbook, inclusive of fumed silica, hydrated silica, silica dimethicone silylate, silica dimethyl silylate, silica silylate, silica captylyl silylate, silica cetyl silylate, fragrances (artificial and natural), dyes and color ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11), adsorbents, lubricants, solvents, moisturizers (including, e.g., emollients, humectants, film formers, occlusive agents, and agents that affect the natural moisturization mechanisms of the skin), water-repellants, UV absorbers (physical and chemical absorbers such as paraaminobenzoic acid (“PABA”) and corresponding PABA derivatives, titanium dioxide, zinc oxide, etc.), essential oils, vitamins (e.g. A, B, C, D, E, and K), trace metals (e.g. zinc, calcium and selenium), anti-irritants (e.g. steroids and non-steroidal anti-inflammatories), botanical extracts (e.g. aloe vera, chamomile, cucumber extract, ginkgo biloba, ginseng, and rosemary), anti-microbial agents, antioxidants (e.g., BHT and tocopherol), chelating agents (e.g., disodium EDTA and tetrasodium EDTA), preservatives (e.g., methylparaben and propylparaben), pH adjusters (e.g., sodium hydroxide and citric acid), absorbents (e.g., aluminum starch octenylsuccinate, kaolin, corn starch, oat starch, cyclodextrin, talc, and zeolite), skin bleaching and lightening agents (e.g., hydroquinone and niacinamide lactate), humectants (e.g., sorbitol, urea, and manitol), exfoliants, waterproofing agents (e.g., magnesium/aluminum hydroxide stearate), skin conditioning agents (e.g., aloe extracts, allantoin, bisabolol, ceramides, dimethicone, hyaluronic acid, and dipotassium glycyrrhizate), or a combination of two or more thereof. In certain embodiments, for example, the lip balm composition may comprise in the range of 1-5 wt. % of the one or more cosmetic ingredients, relative to the total weight of the lip balm composition, for example 5-10 wt. %, 10-15 wt. %, 15-20 wt. %, 20-25 wt. %, 25-30 wt. %, 30-35 wt. %, or the lip balm composition may comprise in the range of 35-40 wt. % of the one or more cosmetic ingredients, relative to the total weight of the lip balm composition. In certain embodiments, for example, the lip balm composition may comprise 1 wt. % of the one or more cosmetic ingredients, relative to the total weight of the lip balm composition, for example, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, 30 wt. %, 31 wt. %, 32 wt. %, 33 wt. %, 34 wt. %, 35 wt. %, 36 wt. %, 37 wt. %, 38 wt. %, 39 wt. %, or the lip balm composition may comprise 40 wt. % of the one or more cosmetic ingredients, relative to the total weight of the lip balm composition.

In certain embodiments, for example, the lip balm composition may contain 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1.1 wt. %, 1.2 wt. %, 1.3 wt. %, 1.4 wt. %, 1.5 wt. %, 1.6 wt. %, 1.7 wt. %, 1.8 wt. %, 1.9 wt. %, 2.1 wt. %, 2.2 wt. %, 2.3 wt. %, 2.4 wt. %, 2.5 wt. %, 2.6 wt. %, 2.7 wt. %, 2.8 wt. %, 2.9 wt. %, 3 wt. %, 4 wt. %, 5 wt. %, 6 wt. %, 7 wt. %, 8 wt. %, 9 wt. %, or 10 wt. % fumed silica. In certain embodiments, for example, the lip balm composition may contain in the range of 0.1-0.5 wt. %, 0.5-1 wt. %, 1.1-1.5 wt. %, 1.5-2 wt. %, 2.1-2.5 wt. %, 2.5-3 wt. %, 3-5 wt. %, or 5-10 wt. % fumed silica. In certain embodiments, for example, the lip balm composition may contain in the range of 0.1-2 wt. % fumed silica, for example in the range of 0.5-1.5 wt. %, 0.75-1.25 wt. %, or the lip balm composition may comprise in the range of 0.9-1.1 wt. % fumed silica.

In certain embodiments, for example, the lip balm composition may contain 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1.1 wt. %, 1.2 wt. %, 1.3 wt. %, 1.4 wt. %, 1.5 wt. %, 1.6 wt. %, 1.7 wt. %, 1.8 wt. %, 1.9 wt. %, 2.1 wt. %, 2.2 wt. %, 2.3 wt. %, 2.4 wt. %, 2.5 wt. %, 2.6 wt. %, 2.7 wt. %, 2.8 wt. %, 2.9 wt. %, 3 wt. %, 4 wt. %, 5 wt. %, 6 wt. %, 7 wt. %, 8 wt. %, 9 wt. %, or 10 wt. % silica dimethyl silylate. In certain embodiments, for example, the lip balm composition may contain in the range of 0.1-0.5 wt. %, 0.5-1 wt. %, 1.1-1.5 wt. %, 1.5-2 wt. %, 2.1-2.5 wt. %, 2.5-3 wt. %, 3-5 wt. %, or 5-10 wt. % silica dimethyl silylate. In certain embodiments, for example, the lip balm composition may contain in the range of 0.1-2 wt. % silica dimethyl silylate, for example in the range of 0.5-1.5 wt. %, 0.75-1.25 wt. %, or the lip balm composition may comprise in the range of 0.9-1.1 wt. % silica dimethyl silylate.

Flavorants: In certain embodiments, for example, the lip balm composition may comprise one or more natural or artificial flavorants. In certain embodiments, for example, the one or more natural or artificial flavorants may be artificial tsubaki oil enhancer FZ9255 (citrus), sweetness enhancers such as N/A Sweetness Enhancer FZ7492, sesame, soybean, apricot kernel, palm, peanut, safflower, coconut, olive, cocoa butter, palm kernel, shea butter, sunflower, almond, avocado, borage, carnauba, hazel nut, castor, cotton seed, evening primrose, orange roughy, rapeseed, rice bran, walnut, wheat germ, peach kernel, babassu, mango seed, black current seed, jojoba, macademia nut, sea buckthorn, sasquana, tsubaki, mallow, meadowfoam seed, coffee, emu, mink, grape seed, thistle, tea tree, pumpkin seed, kukui nut, or a combination of two or more thereof. In certain embodiments, for example, the total amount of flavorants may range from 1-2 wt. %, 2-3 wt. %, 3-4 wt. %, 4-5 wt. %, 5-6 wt. %, 6-7 wt. %, 7-8 wt. %, 8-9 wt. %, or 9-10 wt. % by weight of the composition. In certain embodiments, for example, the total amount of flavor may be 1 wt. %, 2 wt. %, 3 wt. %, 4 wt. %, 5 wt. %, 6 wt. %, 7 wt. %, 8 wt. %, 9 wt. %, or 10 wt. % by weight of the composition. In certain embodiments, for example, the lip balm composition may contain 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, or 30 wt. % of one or more flavorants. In certain embodiments, for example, the lip balm composition may contain in the range of 1-5 wt. %, 5-10 wt. %, 10-15 wt. %, 15-20 wt. %, 20-25 wt. %, or 25-30 wt. % of one or more flavorants. In certain embodiments, for example, the lip balm composition may comprise in the range of 0.25-4 wt. % FZ9255, for example in the range of 0.5-2 wt. %, or the lip balm composition may comprise in the range of 0.8-1.2 wt. % FZ9255. In certain embodiments, for example, the lip balm composition may comprise in the range of 0.25-2 wt. % FZ7492, for example in the range of 1-4 wt. %, or the lip balm composition may comprise in the range of 1-3 wt. % FZ7492.

Essential oils: In certain embodiments, for example, the lip balm composition may comprise one or more essential oils. In certain embodiments, for example, the one or more essential oils may be rose oil, peppermint oil, sesame oil, macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, or ylang ylang, or a combination of two or more thereof. In certain embodiments, for example, the lip balm composition may contain in the range of 1-2 wt. %, 2-3 wt. %, 3-4 wt. %, 4-5 wt. %, 5-6 wt. %, 6-7 wt. %, 7-8 wt. %, 8-9 wt. %, or 9-10 wt. % of the one or more essential oils, relative to the total weight of the lip balm composition. In certain embodiments, for example, the lip balm composition may contain 1 wt. %, 2 wt. %, 3 wt. %, 4 wt. %, 5 wt. %, 6 wt. %, 7 wt. %, 8 wt. %, 9 wt. %, or 10 wt. % of the one or more essential oils, relative to the total weight of the lip balm composition.

Antioxidants: In certain embodiments, for example, one or more natural or synthetic antioxidants may be included in the lip balm composition to defend against environmental stress on skin, for example by interrupting oxidation reactions and preventing the effects of oxygen radicals (e.g. peroxides). In certain embodiments, for example, the one or more antioxidants may be acetyl cysteine, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamythydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris (nonylphenyl) phosphate, or a combination of two or more thereof. In certain embodiments, for example, the lip balm composition may contain in the range of 1-2 wt. %, 2-3 wt. %, 3-4 wt. %, 4-5 wt. %, 5-6 wt. %, 6-7 wt. %, 7-8 wt. %, 8-9 wt. %, or 9-10 wt. % of the one or more antioxidants, relative to the total weight of the lip balm composition. In certain embodiments, for example, the lip balm composition may contain 1 wt. %, 2 wt. %, 3 wt. %, 4 wt. %, 5 wt. %, 6 wt. %, 7 wt. %, 8 wt. %, 9 wt. %, or 10 wt. % of the one or more antioxidants, relative to the total weight of the lip balm composition.

UV Sunscreen agents: In certain embodiments, for example, one or more UV absorbing agents may be in included in the lip balm composition to protecting skin from UV radiation (e.g., UVA, UVB, and/or UVC radiation). In certain embodiments, for example, the one or more UV absorbing agents may be one or more of those listed in the international Cosmetic Ingredient Dictionary Handbook, 12th Edition (2008), Vol. 3, pages 3236-3239, which is incorporated by reference. In certain embodiments, for example, the one or more UV absorbing agents may be a chemical and/or physical sunblock. In certain embodiments, for example, the one or more chemical sunblock may be para-aminobenzoic acid (PABA), PABA esters (glyceryl PABA, amyldimethyl PABA and octyldimethyl PABA), butyl PARA, ethyl PABA, ethyl dihydroxypropyl PABA, benzophenones (oxybenzone, sulisobenzone, benzophenone, and benzophenone-1 through 12), cinnamates (and octyl methoxycinnamate, isoamyl p-methoxycinnamate, octylmethoxy cinnamate, cinoxate, diisopropyl methyl cinnamate, DEA-methoxycinnamate, ethyl diisopropylcinnamate, glyceryl octanoate dimethoxycinnamate and ethyl methoxycinnamate), cinnamate esters, salicylates (homomethyl salicylate, benzyl salicylate, glycol salicylate, isopropylbenzyl salicylate, etc.), anthranilates, ethyl urocanate, homosalate, dibenzoylmethane derivatives (e.g., avobenzone), styrene/acrylates copolymer (e.g., SUNSPHERES™ sold by Rohm and Haas), octocrylene, and combinations of two or more thereof. In certain embodiments, for example, the one or more physical sunblocks may kaolin, talc, petrolatum, metal oxides (e.g., titanium dioxide and zinc oxide), or a combination of two or more thereof. In certain embodiments, for example, the lip balm composition may contain in the range of 1-2 wt. %, 2-3 wt. %, 3-4 wt. %, 4-5 wt. %, 5-6 wt. %, 6-7 wt. %, 7-8 wt. %, 8-9 wt. %, or 9-10 wt. % of the one or more UV absorbing agents, relative to the total weight of the lip balm composition. In certain embodiments, for example, the lip balm composition may contain 1 wt. %, 2 wt. %, 3 wt. %, 4 wt. %, 5 wt. %, 6 wt. %, 7 wt. %, 8 wt. %, 9 wt. %, or 10 wt. % of the one or more UV absorbing agents, relative to the total weight of the lip balm composition.

In certain embodiments, for example, the lip balm may comprise Polyamide-8; Polyamide-3, ricinus communis (castor) seed oil; dicaprylyl carbonate; octyldodecanol; fumed silica; decyl isostearate & isostearyl isostearate; butyrospetmum parkii (shea) oil; pentaerythrityl tetraisostearate; and flavorant.

In certain embodiments, for example, the lip balm may comprise one or more of Polyamide-8; Polyamide-3, ricinus communis (castor) seed oil; dicaprylyl carbonate; octyldodecanol; fumed silica; decyl isostearate & isostearyl isostearate; butyrospermum parkii (shea) oil; pentaerythrityl tetraisostearate; and flavorant.

In certain embodiments, for example, the lip balm may consist of Polyamide-8; Polyamide-3, ricinus communis (castor) seed oil; dicaprylyl carbonate; octyldodecanol; fumed silica; decyl isostearate & isostearyl isostearate; butyrospermum parkii (shea) oil; pentaerythrityl tetraisostearate; and flavorant.

In certain embodiments, for example, the lip balm may comprise by weight between 1-10 wt. % Polyamide-8; 1-20 wt. % Polyamide-3, 1-25 wt. % ricinus communis (castor) seed oil; 1-14 wt. % dicaprylyl carbonate; 1-10 wt. % octyldodecanol; 0.5-5 wt. % fumed silica; 1-20 wt. % decyl isostearate & isostearyl isostearate; 1-15 wt. % butyrospermum parkii (shea) oil; 1-15 wt. % pentaerythrityl tetraisostearate; and 1-10 wt. % flavorant.

In certain embodiments, for example, the lip balm may comprise by weight between 3-8 wt. % Polyamide-8; 13-18 wt. % Polyamide-3, 18-23 wt. % ricinus communis (castor) seed oil; 12-17 wt. % dicaprylyl carbonate; 2-7 wt. % octyldodecanol; 0.5-2 wt. % fumed silica; 13-18 wt. % decyl isostearate & isostearyl isostearate; 8-13 wt. % butyrospermum parkii (shea) oil; 9-14 wt. % pentaerythrityl tetraisostearate; and 2-7 wt. % flavorant.

In certain embodiments, for example, the lip balm may comprise by weight 6 wt. % Polyamide-8; 15 wt. % Polyamide-3, 20 wt. % ricinus communis (castor) seed oil; 14 wt. % dicaprylyl carbonate; 4 wt. % octyldodecanol; 1 wt. % fumed silica; 15 wt. % decyl isostearate & isostearyl isostearate; 10 wt. % butyrospermum parkii (shea) oil; 11 wt. % pentaerythrityl tetraisostearate; and 4 wt. % flavorant.

In certain embodiments, for example, the lip balm may consist of by weight between 1-10 wt. % Polyamide-8; 1-20 wt. % Polyamide-3, 1-25 wt. % ricinus communis (castor) seed oil; 1-14 wt. % dicaprylyl carbonate; 1-10 wt. % octyldodecanol; 0.5-5 wt. % fumed silica; 1-20 wt. % decyl isostearate & isostearyl isostearate; 1-15 wt. % butyrospermum parkii (shea) oil; 1-15 wt. % pentaerythrityl tetraisostearate; and 1-10 wt. % flavorant.

In certain embodiments, for example, the lip balm may consist of by weight between 3-8 wt. % Polyamide-8; 13-18 wt. % Polyamide-3, 18-23 wt. % ricinus communis (castor) seed oil; 12-17 wt. % dicaprylyl carbonate; 2-7 wt. % octyldodecanol; 0.5-2 wt. % fumed silica; 13-18 wt. % decyl isostearate & isostearyl isostearate; 8-13 wt. % butyrospermum parkii (shea) oil; 9-14 wt. % pentaerythrityl tetraisostearate; and 2-7 wt. % flavorant.

In certain embodiments, for example, the lip balm may consist of by weight 6 wt. % Polyamide-8; 15 wt. % Polyamide-3, 20 wt. % ricinus communis (castor) seed oil; 14 wt. % dicaprylyl carbonate; 4 wt. % octyldodecanol; 1 wt. % fumed silica; 15 wt. % decyl isostearate & isostearyl isostearate; 10 wt. % butyrospermum parkii (shea) oil; 11 wt. % pentaerythrityl tetraisostearate; and 4 wt. % flavorant.

In certain embodiments, the lip balm may comprise polyamide-8; polyamide-3, ricinus communis (castor) seed oil; dicaprylyl carbonate; octyldodecanol; silica dimethyl silylate; decyl isostearate & isostearyl isostearate; butyrospermum parkii (shea); pentaerythrityl tetraisostearate; cocos nucifera (coconut) oil; helianthus annuus (sunflower) seed oil; aloe barbadensis leaf juice; avocado oil; and flavorant.

In certain embodiments, for example, the lip balm may comprise one or more of polyamide-8; polyamide-3, ricinus communis (castor) seed oil; dicaprylyl carbonate; octyldodecanol; silica dimethyl silylate; decyl isostearate & isostearyl isostearate; butyrospermum parkii (shea) oil; pentaerythrityl tetraisostearate; cocos nucifera (coconut) oil; helianthus annuus (sunflower) seed oil; aloe barbadensis leaf juice; avocado oil; and flavorant.

In certain embodiments, for example, the lip balm may consist of polyamide-8; polyamide-3, ricinus communis (castor) seed oil; dicaprylyl carbonate; octyldodecanol; silica dimethyl silylate; decyl isostearate & isostearyl isostearate; butyrospermum parkii (shea) oil; pentaerythrityl tetraisostearate; cocos nucifera (coconut) oil; helianthus annuus (sunflower) seed oil; aloe barbadensis leaf juice; avocado oil; and flavorant.

In certain embodiments, for example, the lip balm may comprise by weight between 1-10 wt. % polyamide-8; 10-20 wt. % polyamide-3, 10-30 wt. % ricinus communis (castor) seed oil; 1-20 wt. % dicaprylyl carbonate; 1-10 wt. % octyldodecanol; 0.1-5 wt. % silica dimethyl silylate; 10-25 wt. % decyl isostearate & isostearyl isostearate; 1-20 wt. % butyrospermum parkii (shea) oil; 1-2.0 wt. % pentaerythrityl tetraisostearate; 0.05-5 wt. % cocos nucifera (coconut) oil; 0.05-5 wt. % helianthus annuus (sunflower) seed oil and aloe barbadensis leaf juice; 0.05-5 wt. % avocado oil; and 1-10 wt. % flavorant.

In certain embodiments, for example, the lip balm may comprise by weight between 2-7 wt. % polyamide-8; 12-17 wt. % polyamide-3, 18-23 wt. % ricinus communis (castor) seed oil; 12-17 wt. % dicaprylyl carbonate; 2-7 wt. % octyldodecanol; 0.1-3 wt. % silica dimethyl silylate; 15-20 wt. % decyl isostearate & isostearyl isostearate; 8-13 wt. % butyrospermum parkii (shea) oil; 8-13 wt. % pentaerythrityl tetraisostearate; 0.05-2 wt. % cocos nucifera (coconut) oil; 0.05-2 wt. % helianthus annuus (sunflower) seed oil and aloe barbadensis leaf juice; 0.05-2 wt. % avocado oil; and 1-5 wt. % flavorant.

In certain embodiments, for example, the lip balm may comprise by weight 5 wt. % polyamide-8; 15 wt. % polyimide-3, 20 wt. % ricinus communis (castor) seed oil; 14 wt. % dicaprylyl carbonate; 4 wt. % octyldodecanol; 1 silica dimethyl silylate; 17 wt. % decyl isostearate & isostearyl isostearate; 10 wt. % butyrospermum parkii (shea) oil; 10.7 wt. % pentaerythrityl tetraisostearate; 0.1 wt. % cocos nucifera (coconut) oil; 0.1 wt. % helianthus annuus (sunflower) seed oil and aloe barbadensis leaf juice; 0.1 wt. % avocado oil; and 3 wt. % flavorant.

In certain embodiments, for example, the lip balm may consist of by weight between 1-10 wt. % polyamide-8; 10-20 wt. % polyimide-3, 10-30 wt. % ricinus communis (castor) seed oil; 1-20 wt. % dicaprylyl carbonate; 1-10 wt. % octyldodecanol; 0.1-5 wt. % silica dimethyl silylate; 10-25 wt. % decyl isostearate & isostearyl isostearate; 1-20 wt. % butyrospermum parkii (shea) oil; 1-20 wt. % pentaerythrityl tetraisostearate; 0.05-5 wt. % cocos nucifera (coconut) oil; 0.05-5 wt. % helianthus annuus (sunflower) seed oil and aloe barbadensis leaf juice; 0.05-5 wt. % avocado oil; and 1-10 wt. % flavorant.

In certain embodiments, for example, the lip balm may consist of by weight between 2-7 wt. % polyamide-8; 12-17 wt. % polyimide-3, 18-23 wt. % ricinus communis (castor) seed oil; 12-17 wt. % dicaprylyl carbonate; 2-7 wt. % octyldodecanol; 0.1-3 wt. % silica dimethyl silylate; 15-20 wt. % decyl isostearate & isostearyl isostearate; 8-13 wt. % butyrospermum parkii (shea) oil; 8-13 wt. % pentaerythrityl tetraisostearate; 0.05-2 wt. % cocos nucifera (coconut) oil; 0.05-2 wt. % helianthus annuus (sunflower) seed oil and aloe barbadensis leaf juice; 0.05-2 wt. % avocado oil; and 1-5 wt. % flavorant.

In certain embodiments, for example, the lip balm composition may be clear, transparent, or translucent. The terms “transparent,” and “translucent” may be understood by the conventional definitions given in the dictionary. Thus, a translucent composition allows light to pass through without, however, allowing the contours of objects to be sharply distinguished. A transparent composition allows light to pass through easily and allows objects to be sharply distinguished through its thickness. In certain embodiments, for example, transparency may be measured according to the Transmittance Test.

In certain embodiments, for example, the lip balm composition may have an initial transmittance of greater than 70% according to the Transmittance Test, for example an initial transmittance of greater than 75%, greater than 80%, greater than 85%, greater than 90%, greater than 91%, greater than 92%, greater than 93%, greater than 94%, greater than 95%, greater than 96%, greater than 97%, greater than 98%, greater than 99%, or an initial transmittance of greater than 99.5% according to the Transmittance Test. In certain embodiments, for example, the lip balm may have an initial transmittance in the range of 70-99.5% according to the Transmittance Test, for example the lip balm may have an initial transmittance in the range of 80-99%, 90-97%, 92-96%, or an initial transmittance in the range of 93.5-95% according to the Transmittance Test. In certain embodiments, for example, In certain embodiments, for example, the lip balm may have an initial transmittance of 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% according to the Transmittance Test.

In certain embodiments, for example, the lip balm may have a transmittance of greater than 70% after 24 hours according to the Transmittance Test, for example a transmittance after 24 hours of greater than 75%, greater than 80%, greater than 85%, greater than 90%, greater than 91%, greater than 92%, greater than 93%, greater than 94%, greater than 95%, greater than 96%, greater than 97%, greater than 98%, greater than 99%, or a transmittance after 24 hours of greater than 99.5% according to the Transmittance Test. In certain embodiments, for example, the lip balm may have a transmittance after 24 hours in the range of 70-99.5% according to the Transmittance Test, for example the lip balm may have a transmittance after 24 hours in the range of 80-99%, 90-97%, 92-96%, or a transmittance after 24 hours in the range of 93.5-95% according to the Transmittance Test. In certain embodiments, for example, the lip balm may have a transmittance after 24 hours of 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% according to the Transmittance Test.

In certain embodiments, for example, the lip balm may have a first moisture reading of at least 60 according to the Moisture Retention Test, for example a first moisture reading of at least 70, at least 80, at least 90, at least 95, at least 100, at least 125, at least 150, at least 175, at least 200, at least 225, or a first moisture reading of at least 250 according to the Moisture Retention Test. in certain embodiments, for example, the lip balm may have a first moisture reading in the range of 100-250 according to the Moisture Retention Test, for example a first moisture reading in the range of 125-200, 150-200, or a first moisture reading in the range of 150-175 according to the Moisture Retention Test.

In certain embodiments, for example, the lip balm may have a second moisture reading, taken at about 15 minutes after the first reading, of at least 50 according to the Moisture Retention Test, for example a second moisture reading of at least 75, at least 80, at least 90, at least 95, at least 125, or a second moisture reading of at least 150 according to the Moisture Retention Test. In certain embodiments, for example, the lip balm may have a second moisture reading, taken at about 15 minutes, after the first reading, according to the Moisture Retention Test, that has a value that is at least 50%, for example at least 55%, 60%, 65%, 70%, or 75% of the Moisture Retention Test value of the first reading. In certain embodiments, for example, the lip balm may have a second moisture reading, taken at about 15 minutes after the first reading, in the range of 50-200 according to the Moisture Retention Test, for example a second moisture reading in the range of 75-150, 75-125, or a second moisture reading in the range of 90-120 according to the Moisture Retention Test.

In certain embodiments, for example, the lip balm may have a payoff of at least 0.1 grams according to the Payoff Test, for example a payoff of at least 0.2 grams, at least 0.3 grams, at least 0.4 grams, or a payoff of at least 0.5 grams according to the Payoff Test. In certain embodiments, for example, the lip balm may have a payoff in the range of 0.1-1.0 grams according to the Payoff Test, for example a payoff in the range of 0.2-0.75 grams, in the range of 0.25-0.6 grams, in the range of 0.2-0.6, or a payoff in the range of 0.3-0.5 grams according to the Payoff Test.

In certain embodiments, for example, the lip balm may have a glide force of less than 10 according to the Payoff Test, for example a glide force of less than 8, less than 6, or a glide force of less than 5 according to the Payoff Test. In certain embodiments, for example, the lip balm may have a glide force in the range of 3-10 according to the Payoff Test, for example a glide force in the range of 4-8, in the range of 5-7, or a glide force in the range of 6-7 according to the Payoff Test.

In certain embodiments, for example, the lip balm may have a hardness in the range of 5-30 mm according to the Hardness Test, for example a hardness in the range of 10-25 mm, in the range of 15-25 mm, a hardness in the range of 16-22 mm, a hardness in the range of 16-19 mm, a hardness in the range of 20-22 mm, or a hardness in the range of 18-20 mm according to the Hardness Test. In certain embodiments, for example, the lip balm may have a hardness in the range of 16-24 mm when measured using ASTM Standard D1321-10, for example a hardness in the range of 1-8 mm, in the range of 8-16 mm, in the range of 16-24 mm, or the lip balm may have a hardness in the range of 24-32 mm when measured using ASTM Standard D1321-10.

### EXAMPLES

A series of clear lip balm formulations were prepared having the compositions shown in Table 1.

The lip balm composition of Example 1 consisted of 5 wt. % polyamide-8; 15 wt. % polyamide-3, 20 wt. % ricinus communis (castor) seed oil; 14 wt. % dicaprylyl carbonate; 4 wt. % octyldodecanol; 1 silica dimethyl silylate; 17 wt. % decyl isostearate & isostearyl isostearate; 10 wt. % butyrospermum parkii (shea) oil; 10.7 wt. % pentaerythrityl tetraisostearate; 0.1 wt. % cocos nucifera (coconut) oil; 0.1 wt. % helianthus annuus (sunflower) seed oil and aloe barbadensis leaf juice; 0.1 wt. % persea gratissima (avocado) oil; and 3 wt. % flavorants (1 wt. % flavor FZ9255 (citrus) and 2 wt. % N/A Sweetness Enhancer FZ7492). To prepare the lip balm, required quantities of dicaprytyl carbonate, decyl carbonate, isostearyl isostearate, pentaerythrityl tetraisostearate, and butyrospermum parkii (shea) oil were heated to a temperature in the range of 90-95° C. (194-203° F.) and blended at medium speed (without forming a vortex) in a kettle. The silica dimethyl silylate was next added to the kettle and the rate of mixing increased to create a vortex. After ten minutes, the polyamide-3 and polyamide-8 were added to the kettle and the batch mixed for an additional ten minutes. Heat was then reduced and the castor oil added to the kettle, whereby the batch temperature was reduced to a temperature in the range of 78-82° C. (172-180° F.). Next, the octyldodecanol, cocos nucifera oil, helianthus annuus seed oil, aloe barbadensis leaf juice, persea gratissima oil, Flavor FZ9255 and N/A Sweetness Enhancer FZ7492 were added to the batch, and the batch mixed (to form a vortex) until ready to pour.

Example 1 was tested according to the methods described below for transmittance, moisture retention, payoff, and temperature stability. Results are given in Table 2. The details of temperature stability testing are given in Table 3.

TABLE 1
Lip Balm Compositions

| | | | | | | Comparative |
|---|---|---|---|---|---|---|
| Component | Example 1 | Example 2 | Example 3 | Example 4 | Example 5 | Example |
| Polyamide-8 | 5% | 6% | 5% | 7% | 6% | 20% |
| Polyamide-3 | 15% | 15% | 15% | 15% | 15% | 10% |
| Ricinus communis | 20% | 20% | 20% | 20% | 20% | 8% |
| (castor) seed oil | | | | | | |
| Dicaprylyl carbonate | 14% | 14% | 49% | 47% | 49% | — |
| Octyldodecanol | 4% | 4% | — | 4% | 4% | 22% |
| Fumed silica | — | 1% | 1% | 1% | 1% | — |
| Silica dimethyl | 1% | — | — | — | — | — |
| silylate | | | | | | |
| Decyl isostearate & | 17% | 15% | 10% | 6% | 5% | 10% |
| isostearyl isostearate | | | (isostearyl | (isostearyl | (isostearyl | (isostearyl |
| | | | isostearate | isostearate | isostearate | isostearate |
| | | | only) | only) | only) | only) |
| Butyrospermum parkii | 10% | 10% | — | — | — | — |
| (Shea) Oil | | | | | | |
| Pentaerythrityl | 10.7% | 11% | — | — | — | — |
| tetraisostearate | | | | | | |
| PPG-3 Benzyl ether | — | — | — | — | — | 10% |
| myristate | | | | | | |
| Isostearyl alcohol | — | — | — | — | — | 10% |
| Octyldodecyl/PPG-3 | — | — | — | — | — | 5% |
| myristyl ether | | | | | | |
| dimer dilinoleate | | | | | | |
| Cocos nucifera | 0.1% | — | — | — | — | — |
| (coconut) oil | | | | | | |
| Helianthus annuus | .1% | — | — | — | — | — |
| (sunflower) seed oil (and) | | | | | | |
| aloe barbadensis leaf juice | | | | | | |
| Avocado oil | .1% | — | — | — | — | — |
| Flavorant(s) | 3% | 4% | — | — | — | 5% |

TABLE 2
Lip Balm Tests

| | | | | eos Visibly | Comparative | Beeswax |
|---|---|---|---|---|---|---|
| Test | Example 1 | ChapStick | eos Organic | Soft | Example | formula |
| Transmittance Test | | | | | | |
| 10 minutes post heating | 94.1% | | | | | |
| 24 hours post heating | 93.5% | | | | | |
| Moisture Retention Test | | | | | | |
| First Reading | 172 | 130 | 180 | 250 | | |
| Second Reading (15 min.) | 105 | 0 | 100 | 180 | | |
| Payoff Test | | | | | | |
| Payoff (grams) | 0.3-0.5 | | | | | 0.1-0.3 |
| Glide Force | 6.5 | | | | 10.2 | 5.7 |

TABLE 3
Temperature Stability Testing of
Lip Balm Composition of Example 1

| | 1 day | 1 week | 2 week | 3 week | 1 month | 3 months |
|---|---|---|---|---|---|---|
| Appearance | | | | | | |
| Test | | | | | | |
| 40° C. | Passed | Passed | Not | Passed | Passed | Passed |
| | | | Tested | | | |
| 50° C. | Passed | Passed | Passed | Passed | Passed | Passed |
| Color Test | | | | | | |
| Freeze-Thaw | Passed | Passed | Not | Passed | Passed | Passed |
| | | | Tested | | | |
| 4° C. | Passed | Passed | Not | Passed | Passed | Passed |
| | | | Tested | | | |
| 25° C. | Passed | Passed | Not | Passed | Passed | Passed |
| | | | Tested | | | |
| 40° C. | Passed | Passed | Not | Passed | Passed | Passed |
| | | | Tested | | | |
| 50° C. | Passed | Passed | Passed | Passed | Passed | Passed |
| Odor Test | | | | | | |
| Freeze-Thaw | Passed | Passed | Not | Passed | Passed | Passed |
| | | | Tested | | | |
| 4° C. | Passed | Passed | Not | Passed | Passed | Passed |
| | | | Tested | | | |
| 25° C. | Passed | Passed | Not | Passed | Passed | Passed |
| | | | Tested | | | |
| 40° C. | Passed | Passed | Not | Passed | Passed | Passed |
| | | | Tested | | | |
| 50° C. | Passed | Passed | Passed | Passed | Passed | Passed |
| Texture Test | | | | | | |
| Freeze-Thaw | Passed | Passed | Not | Passed | Passed | Passed |
| | | | Tested | | | |
| 4° C. | Passed | Passed | Not | Passed | Passed | Passed |
| | | | Tested | | | |
| 25° C. | Passed | Passed | Not | Passed | Passed | Passed |
| | | | Tested | | | |
| 40° C. | Passed | Passed | Not | Passed | Passed | Passed |
| | | | Tested | | | |
| 50° C. | Passed | Passed | Passed | Passed | Passed | Passed |
| Packaging | | | | | | |
| Test | | | | | | |
| Freeze-Thaw | Passed | Passed | Not | Passed | Passed | Passed |
| | | | Tested | | | |
| 4° C. | Passed | Passed | Not | Passed | Passed | Passed |
| | | | Tested | | | |
| 25° C. | Passed | Passed | Not | Passed | Passed | Passed |
| | | | Tested | | | |
| 40° C. | Passed | Passed | Not | Passed | Passed | Passed |
| | | | Tested | | | |
| 50° C. | Passed | Passed | Passed | Passed | Passed | Passed |
| Hardness | | | | | | |
| Test | | | | | | |
| 25° C. | Passed | Passed | Passed | Passed | Passed | Passed |
| Droop Test | | | | | | |
| 50° C. | Passed | Passed | Passed | Passed | Passed | Passed |

Transmittance Test: 20 mL lip balm samples were heated to 80° C., transferred to quartz cuvettes, and allowed to cool and solidify for 10 minutes. Transmittance measurements were then made at a wavelength of 600 nm using a Thermoscientific Genesys 10 s UV-Vis Spectrophotometer and repeated 24 hours thereafter.

Moisture Retention Test: three (3) drops of water were applied to a paper towel, followed by the application of the lip balm composition to the top side of the towel. The top side of the paper towel was then dried with a blow dryer for five (5) minutes, and moisture readings taken from the back side of the towel using a Nova DPM 9003 BT moisture meter. A second set of readings were taken 15 minutes thereafter (no additional blow drying).

Payoff Test: a two (2)-inch diameter cylinder of lip balm was moved in a 5 cm cycle (5 cm followed by 5 cm in reverse direction) against a surface at a speed of 1 cm/sec for five cycles using a cantilever apparatus that presses the lip balm against the surface. The test was repeated three times on fresh surfaces. Payoff was measured as the mass of lip balm transferred. Glide force was also measured in this same apparatus.

Temperature Stability Testing: four (4) ounce samples and finish product samples of the lip balm composition of Example 1 were tested for appearance, color, odor, texture, packaging, hardness, and droop under the temperature conditions and durations specified in Table 3. Freeze-thaw conditions comprised cooling a lip balm sample to a temperature of −10° C. for 24 hours, followed by thawing for 24 hours to 25° C. The cooling and thawing were repeated for three (3) cycles and measurements made after each cycle.

Appearance Test: visual observations were made to detect whether the lip balm sample exhibited sweating (syneresis). Two droplets or more of sweating were recorded as a failure.

Color Test: visual observations were made to detect whether the lip balm maintained a clear color in the range of D-J according to the GIA International Diamond Grading System™. An observation outside the aforementioned range was recorded as a failure.

Odor Test: sniff observations were made at a distance of about 0.5 inches from the lip balm sample to detect whether the lip balm maintained an odor compared to control (i.e., a new sample at 4° C.). A measurement of less than 3 on a scale of 1-5 was recorded as a failure, wherein a value of 5 represented the odor of a new sample of the lip balm at 4° C. and a value of 1 represented no detectable odor (versus control).

Texture Test: visual observations were made to detect whether the lip balm maintained a smooth texture. A measurement of less than 3 on a scale of 1-5 was recorded as a failure, wherein a value of 5 represented the appearance of a new sample of the lip balm at 25° C. and a value of 1 represented a completely frosted appearance.

Packaging Test: observations were made to determine if a packaged lip balm sample matched the appearance, color, odor, and texture of four (4) ounce and finished product lip balm samples tested under 25° C. constant temperature conditions.

Hardness Test: measurements were made to determine whether the hardness of the lip balm samples fell within the range of 16-24 mm. Each hardness measurement was an average of three measurements taken with a Humboldt H-1240 penetrometer equipped with an H1280 needle (weight of 2.6 grams), wherein the needle was allowed to penetrate the lip balm under the force of gravity for five seconds per measurement.

Droop Test (or self-supporting test): finished product samples were oriented at +90°, −90°, or 180° relative to the upright position to determine whether the finished product samples experienced any deformation. Upon initial set-up, the lowest point of the lip balm mound was recorded and again at the noted time. A deformation larger than 2 mm (measured as the lowest point of the lip balm minus the lowest initial point of the lip balm) was recorded as a failure.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in sonic cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The features disclosed in this specification (including accompanying claims, abstract, and drawings) may be replaced by alternative features serving the same, equivalent or similar purpose, unless expressly stated otherwise. Thus, unless expressly stated otherwise, each feature disclosed is one example of a generic series of equivalent or similar features.

The subject headings used in the detailed description are included for the ease of reference of the reader and should not be used to limit the subject matter found throughout the disclosure or the claims. The subject headings should not be used in construing the scope of the claims or the claim limitations.

The disclosure has been described with reference to particular embodiments. However, it will be readily apparent to those skilled in the art that it is possible to embody the disclosure in specific forms other than those of the embodiments described above. The embodiments are merely illustrative and should not be considered restrictive. The scope of the disclosure is given by the appended claims, rather than the preceding description, and all variations and equivalents that fall within the range of the claims are intended to be embraced therein.

What is claimed is:

1. A lip balm applicator comprising:
i) a lower portion comprising a support structure contained, at least partially, within the lower portion;
ii) an upper portion removably connected to at least a part of the lower portion; and
iii) a mound of transparent lip balm in contact with at least one surface of the lower portion, wherein an application surface of the mound of transparent lip balm is exposed upon removal of the upper portion.

2. The lip balm applicator of claim 1, wherein the lower portion has no moving parts.

3. The lip balm applicator of claim 1, wherein the lip balm has a hardness in the range of 16-24 mm according to the Hardness Test.

4. The lip balm applicator of claim 1, wherein the lip balm has a transmittance of at least 80% after 24 hours according to the Transmittance Test.

5. The lip balm applicator of claim 1 wherein the lip balm has a payoff in the range of 0.2-0.6 grams according to the Payoff Test.

6. A lip balm composition comprising:
i) 60-85 wt. % of at least one emollient;
ii)
iii) less than 40 wt. % of at least one structuring polymer, the lip balm having:
a) a hardness in the range of 16-24 mm according to the Hardness Test;
b) a transmittance of at least 80% after 24 hours according to the Transmittance Test;
c) a payoff in the range of 0.2-0.6 grams according to the Payoff Test; and
d) a moisture retention of at least 70 after 15 minutes according to the Moisture Retention Test

7. The lip balm composition of claim 6, wherein the lip balm composition comprises less than 40 wt. % of any structuring agents.

8. The lip balm composition of claim 6, wherein the lip bairn composition comprises less than 30 wt. % polyamides.

9. The lip balm composition of claim 6, wherein the lip balm composition comprises less than 30 wt. % dicaprylyl carbonate.

10. The lip balm composition of claim 6, wherein the lip balm composition is anhydrous.

11. The lip balm composition of claim 6, wherein the lip balm composition is preservative free.

12. The lip balm composition of claim 6, wherein the lip balm composition is volatile alcohol-free.

13. The lip balm composition of claim 9, comprising at least 10 wt. % and less than 30 wt. % dicaprylyl carbonate.

14. The lip balm composition of claim 6, wherein the lip balm composition is self-supporting, at room temperature.

15. A lip balm applicator comprising:
i) an enclosure comprising:
a) a lower portion comprising a support structure, the support structure contained at least partially within the lower portion; and
b) an upper portion removably connected to at least a part of the lower portion; and
ii) a mound of transparent lip balm enclosed within the enclosure, the mound of transparent lip balm having:
a) an application surface extending from and exposed beyond the lower portion, the application surface having an application surface area of greater than 55% of the total surface area of the mound of transparent lip balm; and
b) a transmittance of at least 85% after 24 hours according to the Transmittance Test.

16. The applicator of claim 15, wherein at least a portion of a support surface of the mound of transparent lip balm is in contact with the support structure.

17. The applicator of claim 15, wherein the mound of transparent lip balm is in fixed relation with the lower portion.

18. The applicator of claim 17, wherein the mound of transparent lip balm is non-telescoping with respect to the enclosure.

19. The applicator of claim 17, wherein the lower portion of the applicator has no moving parts.

20. The applicator of claim 19, wherein the mound of transparent lip balm has:
i) a passing score according to the Droop Test after 1-week at 50° C.;
ii) a payoff in the range of 0.2-0.6 grams according to the Payoff Test; and
iii) a moisture retention of at least 70 after 15 minutes according to the Moisture Retention Test.
